(19) United States
(12) Patent Application Publication
(10) Pub. No.: US 2022/0074923 A1
(43) Pub. Date: Mar. 10, 2022

(54) METHODS OF IDENTIFYING PARKIN-MEDIATED MITOPHAGY ACTIVATING AGENTS

(71) Applicant: THE GOVERNING COUNCIL OF THE UNIVERSITY OF TORONTO, Toronto (CA)

(72) Inventors: Angus George McQuibban, Toronto (CA); Natalia Moskal, Toronto (CA); Peter N. Lewis, Toronto (CA)

(21) Appl. No.: 17/416,305

(22) PCT Filed: Dec. 18, 2019
(86) PCT No.: PCT/CA2019/051852

§ 371 Date: Jun. 18, 2021

Related U.S. Application Data
(60) Provisional application No. 62/781,045, filed on Dec. 18, 2018.

(51) Int. Cl. G01N 33/50

(52) U.S. Cl. CPC G01N 33/5035

(57) ABSTRACT

This disclosure provides a screening method to identify Parkin-mediated mitophagy activating agents wherein the method comprises contacting cells exogenously expressing labelled mutant Parkin polypeptide with a test agent and a mitophagy inducing agent, and measuring the amount of Parkin polypeptide recruited to mitochondria in the cells. Also provided is a kit comprising a plasmid, recombinant cells, a multi-well plate, a pintool head, cells expressing wildtype Parkin, and transfection reagents.

### RELATED FAMILY DETAILS

This PCT Application claims the benefit of U.S. provisional patent application Serial No, 62,781,045 filed on Dec. 18, 2018, which is incorporated by reference in its entirety.

### FIELD

This disclosure generally relates to screening assays for identifying small molecules that improve degradation of defective or damaged mitochondria and more particularly to high through put screening assays for identifying potential drugs that improve degradation of defective or damaged mitochondria.

### BACKGROUND

The survival of dopaminergic neurons depends on the ability to mitigate mitochondrial damage. Mutations in PINK1 and PARK2, the genes encoding the key mediators of a process targeting damaged mitochondria for degradation termed mitophagy, cause an early-onset form of familial Parkinson's disease (PD)^{1,2}. Dysfunction in these proteins has also been observed in sporadic PD. By amplifying the ability to degrade damaged mitochondria, it may be possible to prevent the degeneration of dopaminergic neurons and to delay disease progression.

Parkin is the E3 ubiquitin ligase downstream of PINK1 in this mitophagy pathway. Following mitochondrial damage, PINK1 is stabilized on the outer mitochondrial membrane. Several PINK1-mediated phosphorylation events lead to recruitment of Parkin from the cytosol to the mitochondria. Parkin then designates substrates for proteasomal or autophagic degradation, by tagging them with ubiquitin. Several RNAi-based screens aimed at identifying upstream modulators of Parkin-mediated mitophagy have utilized the subcellular transition undergone by Parkin as a readout³⁻⁵. These efforts have led to the identification of novel regulators of Parkin activity including TOMM7, HSPA1L, BAG4, SIAH3, ATPFIF1 and HK2.

### SUMMARY

Molecules affecting the levels and/or activity of Parkin-mediated mitophagy regulators and others that have yet to be identified may represent potential activators or inhibitors of this neuroprotective pathway. High throughput small molecule screening efforts led to the identification of several compounds that converged on a common canonical target: Rho-associated protein kinase (ROCK).

In the present disclosure, assays aimed at identifying Parkin mediated mitophagy activating and inhibiting agents are provided.

Accordingly, the present disclosure provides in one aspect a screening method comprising:
contacting cells expressing Parkin polypeptide optionally having a wildtype or mutant Parkin polypeptide sequence, with a test agent (TA);
contacting the cells with a mitophagy inducing agent (MIA); and
measuring an amount of Parkin polypeptide recruited to mitochondria in the cells contacted with the test agent compared to control cells treated with a control agent and MIA,
wherein the amount of Parkin polypeptide recruited to mitochondria in the cells contacted with the test agent compared to control cells treated with a control agent and MIA indicates whether the test agent is a putative Parkin mediated mitophagy activating or inhibiting agent.

In an embodiment, the cells are expressing recombinant labelled Parkin polypeptide comprising a wildtype or mutant Parkin polypeptide sequence and a label.

In an embodiment, the cells express endogenous Parkin polypeptide, optionally having a wildtype or mutant Parkin polypeptide sequence, wherein the amount of Parkin polypeptide recruited to mitochondria is measured immunologically using an anti-Parkin antibody.

In an embodiment, the amount comprises determining the percentage of cells with Parkin recruited to mitochondria.

In another aspect, the present disclosure provides a high-throughput screening assay comprising
preparing a plurality of wells comprising test wells and control wells each comprising cells expressing Parkin polypeptide comprising a wildtype or mutant Parkin polypeptide sequence, optionally wherein the Parkin polypeptide is labelled Parkin polypeptide comprising a wildtype or mutant Parkin polypeptide sequence and a label,
contacting the plurality of test wells with a plurality of test agents (TAs) and optionally one or more of the control wells with a control agent,
contacting a subset of the plurality of test wells and control wells with either a mitophagy inducing agent (MIA) or a mitophagy vehicle control,
measuring an amount of Parkin polypeptide recruited to mitochondria in the plurality of test wells and the control wells,
comparing the amount of Parkin polypeptide recruited to mitochondria in the plurality of test wells contacted with TA and MIA to the amount of Parkin polypeptide recruited to mitochondria in the control wells treated with or MIA and optionally a control agent, wherein
a TA that increases Parkin recruitment to the mitochondria compared to control wells is a putative Parkin mediated mitophagy activating agent, and
a TA that decreases Parkin recruitment to the mitochondria compared to control wells is a putative Parkin mediated mitophagy inhibiting agent.

In an embodiment, the high-throughput screening assay comprises using cells recombinantly expressing labelled Parkin, optionally wherein the labelled Parkin comprises a mutant Parkin polypeptide sequence and a label.

In an embodiment, the high-throughput screening assay comprises using cells expressing endogenous Parkin.

In another aspect, the present disclosure provides a kit comprising:
a vector such as a plasmid, comprising a Parkin expression cassette for expressing labelled Parkin polypeptide comprising a wildtype or mutant Parkin polypeptide sequence and a label; and/or
cells expressing wild type or mutant Parkin polypeptide, optionally recombinant cells comprising a mutant Parkin expression cassette for expressing labelled Parkin polypeptide comprising mutant Parkin polypeptide sequence and a label; and
one or more of
a multi-well plate,
a pintool head for delivering small volumes,
a vector such as a plasmid, comprising a wildtype Parkin expression cassette for expressing labelled wildtype Parkin polypeptide,
recombinant cells comprising a wildtype Parkin expression cassette for expressing a labelled wildtype Parkin polypeptide, and one or more transfection or transduction reagents.

In an embodiment, the amount of Parkin polypeptide recruited is assessed by determining the amount (e.g. percentage) of the cells in which Parkin polypeptide is recruited to the mitochondria relative to control cells.

Other features and advantages of the present disclosure will become apparent from the following detailed description. It should be understood, however, that the detailed description and the specific Examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

Embodiments are described below in relation to the drawings in which:

FIG. 1 shows ROCK inhibitors increase Parkin recruitment to damaged mitochondria. (a) Average activity (% of cells with mitochondrial Parkin distribution) of all compounds screened. Based on these values, compounds are classified as either activators samples or inhibitors). ROCK inhibitors Y27632, Y39983 and SR3677 are highlighted. (b) Enhancers of Parkin recruitment from (a) arranged according to common protein target. ROCK inhibitors Fasudil, rho kinase inhibitor II and GSK2699662A. Dashes represent average value amongst each group and pink dots represent activity values for activator compounds whose canonical target is not ROCK. (c) Hits were rank ordered based on z-score. ROCK inhibitors Y27632, Y39983 and SR3677 are highlighted as the top-ranking hits in the kinase inhibitor library screen. (d) Chemical structures of ROCK inhibitors identified (e) Following incubation in 4 μM of small molecules for 16 hours, mitophagy was induced by 20 μM CCCP treatment. Pictured are cells following 2-hour treatment; (f) GFP Parkin WT cells pre-treated with either DMSO or 0.5 μM SR3677 were treated with 10 μM CCCP for 1 hour. Scale bars, 10 μm. (g) Quantification of the percentage of cells with mitochondrial Parkin. P-values were determined by paired student's t-test, *P-value<0.05. Data is expressed as mean±s.e.m.

FIG. 2 shows SR3677 increases Parkin recruitment and activity. (a) HEK293 GFP Parkin cells were treated with varying doses of SR3677 for 2 hours prior to induction of mitophagy with 10 μM CCCP for one hour. The percentage of cells with mitochondrial Parkin increased in a dose-dependent manner. Fitting of dose response curves to this data yields an EC₅₀ value of 57 nM. ≥422 cells was quantified, n=3 (b) HEK293 GFP Parkin cells treated with either 0.5 μM SR3677 or equal volume of DMSO for 2 hours were incubated in 10 μM CCCP for the indicated duration of time. Cell lysates were harvested, proteins were separated by SDS-PAGE and immunoblotting was performed with anti-Mfn2 and anti-actin antibodies. (c) Ponceau VDAC1 levels in each sample normalized to Ponceau staining. (d) Mfn2/actin levels in CCCP treated cells. (e) Densitometry analysis was performed to quantify Mfn2 and VDAC1 levels in each sample (n=4 independent experiments).

FIG. 3 shows targeting of mitochondria to lysosomes and the clearance of mitochondria from cells was increased by SR3677. (a) HeLa cells were co-transfected with Cerulean-Parkin and RGOMP25, a plasmid containing mCherry and GFP in tandem with the transmembrane segment of the OMM protein OMP25. After 24 hours, cells were pre-treated with either 0.5 μM SR3677 or DMSO for 2 hours prior to induction of mitophagy with 10 μM CCCP treatment, in combination with E-64 and leupeptin for a 6-hour duration. Red only pixels represent mitochondria localized to lysosomes, where GFP signal is quenched. (b) Quantification of the percentage of red-only mitochondrial area divided by the total non-background area following the treatments described in (a). ≥50 cells were included in each treatment group, n=3 (c) HeLa cells stably expressing mito-DsRed and GFP Parkin were pre-treated with either DMSO or 0.5 μM SR3677, followed by 24-hour treatment with 10 μM CCCP or DMSO. (d) Quantification of the percentage of cells that retained mitochondrial signal. Cells positive for mitochondrial signal are indicated by arrows in (c). ≥50 cells per treatment group were included, n=4*P-value<0.05. (e) 7-day old TH-GAL4>UAS-mitoQC male flies were placed into vials containing the indicated treatments. Representative images of the dopaminergic neurons of TH-GAL4>UAS-mitoQC flies following feeding on fly food supplemented with H₂O, 0.5 mM SR3677 (SR) or H₂O/SR3677 combined with 5 mM paraquat (PQ). Scale bars, 10 μm. (f) Quantification of the percentage of red-only mitochondrial area divided by the total non-background area, averaged across 0.8 μm z-stacks. Data is expressed as mean±s.e.m (n=4 independent experiments). P-values were determined by one-tailed paired student's t-test, *P<0.05. Error bars represent s.e.m.

FIG. 4 shows genetic manipulations of ROCK2 mirror effects of ROCK inhibitor. (a) Pictured are HEK293 GFP Parkin cells stably expressing either pLKO.1 empty control vector or shRNA against ROCK2 were treated with 10 μM CCCP for 40 minutes. Immunostaining was performed with anti-ATP5A antibody. (b) Cell lines stably expressing either pLKO.1 empty vector or shRNA against ROCK2 were harvested and separated by SDS-PAGE. Immunoblotting was performed with anti-ROCK2 and anti-actin antibody. (c) Quantification of the percentage of cells with mitochondrial Parkin following treatment with 10 μM CCCP. ≥963 cells per treatment group were included, n=3 (d) HEK293 GFP Parkin cells were transfected with Flag-ROCK2. 24 hours following transfection, cells were treated with 10 μM CCCP. Immunostaining was performed with anti-Flag and anti-Hsp60 antibodies. (e) Untransfected cells and cells transfected with Flag-ROCK2 were harvested and proteins were separated by SDS-PAGE. Immunoblotting was performed using anti-ROCK2 and anti-actin. (f) Quantification of the percentage of untransfected and Flag-ROCK2 transfected cells with mitochondrial Parkin. ≥203 cells per treatment group were included, n=3. *P-value<0.05.

FIG. 5 shows SR3677 activates HK2 and increases the pool of HK2 localized to the mitochondria. (a) SH-SY5Y treated with either 0.5 μM SR3677 or with DMSO were separated into total cell cytosolic and mitochondrial fractions. Fractions were separated by SDS-PAGE and immunoblotting was performed with anti-HK2 and anti-ATP5A antibodies. (b) The proposed mechanism through which SR3677 increases Parkin recruitment. (c) Cells serum-starved for 2 hours were treated with either DMSO or SR3677 for 10 or 30 minutes prior to harvesting. Lysates were separated by SDS-PAGE and immunoblotting was performed with anti-HK2 and anti-actin antibodies. Samples were also run on Phos-tag gels in parallel and immunoblotting was performed with anti-HK2 antibody. (d) Densitometry analysis was performed to quantify HK2 levels in each sample, followed by (e) normalization to mitochondrial marker, CValpha. Average HK2/CValpha ratios are plotted (n=2 independent experiments). (f) Densitometry analysis was performed on the band indicated by the arrow in (c). The average intensity of the indicated band normalized to actin loading control is expressed±s.d (n=3 independent experiments). P-values were determined by paired student's t-test for (b, f), *P<0.05. All error bars represent s.e.m.

FIG. 6 shows SR3677 increases the viability of paraquat-treated neurons. (a) Protocol for differentiation of SH-SY5Y cells and experimental set up. (b) Cell viability measurement, as indicated by ATP levels, normalized to protein concentration, of differentiated SH-SY5Y cells co-treated with 500 μM paraquat in combination with various concentrations of SR3677. Values are normalized to the viability of cells treated with paraquat-alone within each trial. Two technical replicates were included for each treatment (n=4).

FIG. 7 shows the survival and climbing ability of paraquat-treated flies was improved by SR3677 (a and b) 7-day old male Canton(S) flies were fed control (water), 10 mM paraquat, 1 mM SR3677 or paraquat in combination with SR3677. Fresh vials containing standard fly food supplemented with the indicated treatments were changed out every 2 days. The number of flies surviving each day was counted and log-rank tests were performed to determine statistical significance. 20 flies per treatment, n=5 (c) The climbing ability of flies treated as described in (a and bin) was assessed 4 days following initial administration of treatments. The number of flies to climb beyond 9.5 cm every 10 seconds was assessed. The percentage of flies in each treatment to cross this line was determined. The climbing ability of each treatment was normalized to the control group treated with water only. 20 flies per treatment, n=5. (d) The climbing ability of UAS-park^{RNAi} and ddc-GAL4>UAS-park^{RNAi} flies fed either paraquat or paraquat in combination with SR3677. 20 flies per treatment, n=7. (e) Proteins lysates from UAS-park^{RNAi} or tubulin>UAS-park^{RNAi} whole flies were separated by SDS-PAGE. Immunoblotting with anti-Parkin and anti-actin antibody was performed. (f) The climbing ability of TH-GAL flies fed either paraquat or paraquat in combination with SR3677. (g) Survival of heteroplasmic mt:Coll^{T300l} flies fed fly food supplemented with water or 2 mM SR3677 and wild-type Canton(S) flies (n=3 independent experiments). (h) The climbing ability of heteroplasmic mt:Coll^{T300l} flies fed either water or 2 mM SR3677 for 7 days. Log-rank tests were performed to determine p-values for survival analyses (a, b, g), *P<0.05. P-values were determined by one-tailed paired student's t-test for all climbing assays (c-e, h), *P<0.05. Data is expressed as mean±s.e.m. All error bars represent s.e.m

FIG. 8 shows HeLa cells expressing GFP-Parkin WT were treated with either DMSO or 20 μM CCCP. Parkin undergoes a subcellular transition from the cytosol to the mitochondria.

FIG. 9 shows PD-linked Parkin mutants fail to localize to damage mitochondria and display impaired degradation of mitochondrial proteins. A) GFP Parkin WT, K161N, T240R and G430D expressing HEK293 cells were treated with CCCP to induce mitophagy. Immunostaining was performed against the mitochondrial marker CValpha. B) Quantification of the percentage of cells with Parkin localized to mitochondria following 2 hour treatment with either DMSO or CCCP. C) Cells were treated with CCCP for the indicated intervals. Cell lysates were then separated by SDS-PAGE and immunoblotting was performed against the mitochondrial proteins Mfn2, CValpha, VDAC1 and Tom20. Actin was used as a loading control.

FIG. 10 shows screening workflow and image analysis.

FIG. 11 shows rank order of compounds described.

FIG. 12 shows mitophagy inhibitors with common canonical targets.

FIG. 13 shows effects of Ac220 on Parkin recruitment and activity. A) HeLa cells expressing GFP Parkin WT and mito-dsRed were treated with either DMSO, 10 μM CCCP or 4 μM Ac220 in combination with CCCP. B) Cell lysates from HeLa cells pre-treated with either DMSO or 4 μM Ac220 prior to addition of 10 μM CCCP were separated by SDS-PAGE and immunoblotting was performed against Mfn2 and actin. C) Cells were pre-treated with either DMSO, 2 μM Ac220, 1 μM Ac220 prior to addition of 10 μM CCCP. D) Cells were pre-treated with indicated concentrations of Ac220 prior to addition of 10 μM CCCP for 1 hour. Cell lysates were separated by SDS-PAGE and immunoblotting was performed against Mfn2 and actin. E) Densitometry analysis of Mfn2 levels normalized to loading control HK2. Data is presented as mean±s.d. F) HeLa GFP Parkin cells were transfected with HA-PINK1 and pre-treated with either DMSO or Ac220 prior to addition of CCCP and Mg132. G) HeLa GFP Parkin cells and H) SH-SY5Y cells were pre-treated with either DMSO or Ac220 prior to addition of CCCP for the indicated time. Immunoblotting was performed using antibodies against PINK1 and actin.

FIG. 14 shows Parkin distribution in GFP-Parkin WT expressing HEK293 cells treated with DMSO, 4 μM alexidine dihydrochloride or 4 μM pyrvinium pamoate prior to addition of DMSO or CCCP.

FIG. 15 shows HeLa cells expressing GFP Parkin WT and mito-dsRed were treated with either DMSO or 0.5 μM SR3677 for 2 hours.

FIG. 16 shows degradation of Mfn2 is increased by ROCK inhibitor treatment. A) GFP Parkin WT-expressing HEK293 cells were treated with either DMSO, 10 μM Y7632, 10 μM Y39983 or 0.5 μM SR3677 for 2 hours prior to 10 μM CCCP addition. Cell lysates were separated by SDS-PAGE and immunoblotting was performed against Mfn2 and actin. B) Densitometry quantification of blots in (A). Mfn2 levels are normalized to actin and an internal control within each blot (cells treated with DMSO only). P-values were determined by paired student's t-test, *P-value<0.05. Data is expressed as mean±s.e.m.

FIG. 17 shows western blot validation of ROCK2 antibody. Cell lysates from HEK293 parental cell line as well as HEK293 cells following CRISPR-mediated genome editing to knock out ROCK2.

FIG. 18 shows SR3677 rescues viability loss following prolonged mitochondrial depolarization in SH-SY5Y cells. A) SH-SY5Y cells were pre-treated with either DMSO or 0.5 μM SR3677 prior to addition of either DMSO or 10 μM CCCP for 24 hours. Following treatment, cells were washed, fixed with 4% paraformaldehyde and stained with crystal violet dye. B) The OD at 550 nm was measured to assess crystal violet staining.

FIG. 19 shows workflow for testing SR3677 in Drosophila PD mode. Drosophila aged 7 days are fed food supplemented with either water (control), 1 mM SR3677, 10 mM paraquat or SR3677 in combination with paraquat. The survival of flies was recorded each day following administration of the treatments and climbing abilities were tested every 2 days by performing negative geotaxis assays.

FIG. 20 shows fly food was supplemented with either (A) paraquat or (B) SR3677 along with blue dye FD&C Blue Food Dye No. 1.

FIG. 21 shows S2R+ cells were treated with either DMSO, paraquat or SR3677 in combination with paraquat. A) Staining was performed with mitoSOX Red Mitochondrial Superoxide dye. B) Quantification of the fluorescence intensity of the mitoSOX probe in the indicated treatments (n=3). Data is presented as mean±s.e.m. P-values were determined by paired student's t-test, *P-value<0.05.

FIG. 22 shows Z′ values for each small molecule library screened.

FIG. 23 shows principal component analysis of Morgan fingerprints of activator compounds. The proximity of two data points is indicative of the degree of structural similarity between two compounds.

FIG. 24 shows Parkin recruitment inhibitors ordered based on average activity score (% of cells with mitochondrial Parkin) of compounds with a common canonical target. Families of Parkin recruitment inhibitors are listed in ascending order.

FIG. 25 shows SR3677 increases degradation of mitochondrial proteins following induction of mitophagy. HEK293 GFP Parkin cells co-treated with 0.5 μM SR3677 or DMSO and 10 μM CCCP for the indicated time (hours). Cell lysates were separated by SDS-PAGE and immunoblotting was performed to assess turnover of IMM/matrix substrates (A) ATP5A, (B) UQCRC2 and (C) COXIV. Densitometry analysis was performed. Protein levels were normalized to Ponceau. Data is expressed as mean±s.e.m (n=6, 7 and 8 for a, b and c, respectively. 4) P-values were determined by paired student's t-test, *P<0.05.

### DETAILED DESCRIPTION OF THE DISCLOSURE

### I. Definitions

The phrase “control agent” as used herein refers to anything that does not appreciably induce Parkin translocation to mitochondria, and may include for example nothing, or the buffer used to dissolve the test agent. The control agent can be the test agent vehicle control (e.g. the control agent) and/or the mitophagy vehicle control.

The term “Parkin” as used herein refers to the protein product encoded by the PRKN gene, for example as identified as UniProtKB number 060260 or Ensembl number ENSG00000185345, and includes without limitation all known Parkin molecules. The sequences disclosed in said accession numbers are herein incorporated by reference.

The phrase “mitochondria detection agent” as used herein refers to any entity capable of detecting directly or indirectly the location of mitochondria within a cell, for example antibodies against mitochondrial proteins and mitochondria-specific stains.

The phrase “mitophagy vehicle control” as used herein refers to a compound that does not appreciably induce mitophagy, for example dimethyl sulfoxide.

The phrase “mitophagy inducing agent” as used herein refers to any compound capable of inducing mitophagy in a cell, including for example the compounds described in Table 1, preferably protonophores such as CCCP or FCCP.

The phrase “mitophagy inhibiting agent” as used herein refers to any compound capable of inhibiting mitophagy and/or Parkin recruitment in response to a MIA in a cell or population of cells, for example by at least 10% relative to a control, e.g. the percentage of cells with mitochondrial Parkin polypeptide translocation in response to the test agent and CCCP is reduced by at least 10% compared to a control treated with vehicle (e.g. control agent) and CCCP.

The phrase “mitophagy activating agent” as used herein refers to any compound capable of increasing mitophagy and/or Parkin recruitment in response to a MIA in a cell or population of cells, for example by at least 10% relative to a control treated with the MIA. e.g. the percentage of cells with mitochondrial Parkin polypeptide translocation in response to the test agent and CCCP is increased by at least 10% compared to a control treated with vehicle (e.g. control agent) and CCCP.

The phrase “mutant Parkin” as used herein refers to a Parkin polypeptide or nucleic acid that encodes a Parkin polypeptide comprising a mutation that reduces Parkin polypeptide translocation to mitochondria in response to CCCP by at least 10%, at least 15%, at least 20%, at least 25% or more, e.g. the percentage of cells with mitochondrial Parkin polypeptide translocation in response to CCCP is reduced by at least 10%. For example, the mutant Parkin can comprise a mutation selected from G430D, T240R, W403A and K161N. The K161N mutation is in the ring 0 domain, the T240R mutation is in the ring 1 domain, the G240D and the W403A mutations are in the ring 2 domain. Mutant Parkin may comprise a mutation in the ring 0 domain, the ring 1 domain or the ring 2 domain.

The term “pinning” as used herein refers to a technique wherein a pinhead tool comprising a plurality of needles is contacted with a plurality of solutions for transferring an aliquot of each solution contacted to another set of solutions.

The term “SR3677” as used herein means a compound having the formula:

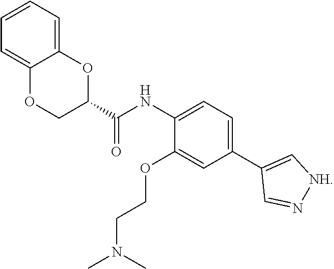 [SMILES: CN(C)CCOc1cc(-c2cn[nH]c2)ccc1NC(=O)[C@@H]1COc2ccccc2O1]

The phrase “suitable level” as used herein with respect to Parkin levels refers to a level of endogenous wildtype Parkin RNA expression that is increased relative to said level in HeLa cells and/or similar to HEK 293 cells, for example as shown in the Human Protein Atlas “RNA Expression Overview” for PARK2 (see for example entry ENSG00000185345 for PARK2 in the Human Protein Atlas, available for example at https://www.proteinatlas.org/ENSG00000185345-PARK2/cell). The suitable level can also be based on endogenous wildtype Parkin polypeptide levels.

The phrase “test agent” as used herein refers to any compound and in particular to a small molecule.

The term “wildtype” as used herein refers to the sequence of a polynucleotide or polypeptide that is most commonly found in individuals of a species.

The phrase “high content imaging” as used herein refers to automated microscopy, fluorescent detection and multi-parameter algorithms to visualize and quantify interactions in cell populations.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

As used in this specification and the appended claims, the singular forms “a”, “an” and “the” include plural references unless the content clearly dictates otherwise. Thus, for example, a composition containing “a cholesterol” includes a mixture of two or more cholesterols. It should also be noted that the term “or” is generally employed in its sense including “and/or” unless the content clearly dictates otherwise.

The definitions and embodiments described in particular sections are intended to be applicable to other embodiments herein described for which they are suitable as would be understood by a person skilled in the art.

### II. Methods

The disclosure relates to screening methods to identify Parkin-mediated mitophagy activating and inhibiting agents.

Accordingly, in one aspect the present disclosure provides a screening method comprising:
contacting cells expressing Parkin polypeptide comprising a wildtype or mutant Parkin polypeptide sequence, with a test agent (TA);
contacting the cells with a mitophagy inducing agent (MIA); and

measuring an amount of Parkin polypeptide recruited to mitochondria in the cells contacted with the test agent compared to control cells treated with a control agent and MIA, wherein the amount of Parkin polypeptide recruited to mitochondria in the cells contacted with the test agent compared to control cells treated with a control agent and MIA indicates whether the test agent is a putative Parkin mediated mitophagy activating or inhibiting agent. In some embodiments, the cells recombinant express labelled Parkin polypeptide comprising a wildtype or mutant Parkin polypeptide sequence and a label.

In an embodiment, the cells recombinantly express labelled Parkin polypeptide comprising a wildtype or mutant Parkin polypeptide sequence and a label and the method comprises:
contacting cells recombinantly expressing labelled Parkin polypeptide comprising a wildtype or mutant Parkin polypeptide sequence and a label, with a test agent (TA);
contacting the cells with a mitophagy inducing agent (MIA); and
measuring an amount of labelled Parkin polypeptide recruited to mitochondria in the cells contacted with the test agent compared to control cells treated with a control agent and MIA,
   wherein the amount of labelled Parkin polypeptide recruited to mitochondria in the cells contacted with the test agent compared to control cells treated with a control agent and MIA indicates whether the test agent is a putative Parkin mediated mitophagy activating or inhibiting agent.

In an embodiment, the cells recombinantly express labelled Parkin polypeptide comprising a mutant Parkin polypeptide sequence and a label.

In one embodiment, the cells express a suitable level of endogenous Parkin. For example in embodiments measuring endogenous Parkin or embodiments measuring both endogenous and recombinantly expressed Parkin, the endogenous level is a suitable level.

In an embodiment, the cells express endogenous Parkin polypeptide, optionally having a wildtype or mutant Parkin polypeptide sequence, wherein the amount of Parkin polypeptide recruited to mitochondria is measured immunologically using an anti-Parkin antibody, optionally fluorescently labelled. For example cells expressing endogenous Parkin can be induced pluripotent stem cells (iPSCs) derived for example from a subject having a wild-type with normal mitophagy or mutant Parkin and reduced mitophagy. In some embodiments, the cells used in the assay have wildtype Parkin and reduced mitophagy for example due to mutation in an upstream or downstream effector.

In an embodiment, the cells expressing endogenous Parkin are iPSCs expressing a mutant Parkin or a wildtype Parkin and have reduced mitophagy.

In an embodiment, the Parkin polypeptide sequence is a mutant Parkin polypeptide sequence. Typically, the method will employ at least labelled Parkin comprising a mutant Parkin sequence. A Parkin polypeptide sequence that is wild type can in some embodiments be used alone if for example the method is for identifying test agents that increase wild type Parkin recruitment. In other embodiments, the assay will involve expressing mutant Parkin and wildtype Parkin. In one embodiment, the Parkin polypeptide recombinantly expressed is mutant Parkin polypeptide. In another embodiment, the Parkin polypeptide recombinantly expressed is a labelled Parkin polypeptide, optionally wherein the label is a fluorescent label. In yet other embodiments, the Parkin polypeptide recombinantly expressed is labelled Parkin comprising a mutant Parkin polypeptide sequence and a label.

In an embodiment, measuring comprises detecting the recombinantly expressed label or the antibody label.

In an embodiment, the measuring further comprises detecting the Parkin, optionally the labelled Parkin polypeptide, in cytosol in the cells. For example, as described herein, whether a cell comprises mitochondrial Parkin can be assessed by assessing Parkin localization in one or a plurality of cellular locations. For example, the measurement can involve measuring multiple locations in the cells, for example at least 2 or at least 3.

Measuring Parkin translocated to the mitochondria can be accomplished using high content microscopy and training the software using samples with cytosolic and/or mitochondrial located Parkin.

Other methods can also be used. For example, the cells can be assayed to determine the localization of a known mitochondrial protein and Parkin, and recruitment can be assessed by measuring colocalization of the detected signals.

In one embodiment, a cell is identified as having or not mitochondrial Parkin by detecting the label (either expressed by the labelled Parkin) or comprised on the antibody used to detect endogenous Parkin) in a plurality of regions of a cell, for example 3 regions. A cell is identified as expressing mitochondrial Parkin based on the assessment of the plurality of regions.

In some embodiments, the plurality of regions comprises cytosolic and mitochondrial regions.

Preferred cells for making the cells recombinantly expressing labelled Parkin express at least a minimal amount of endogenous Parkin. HEK 293 cells and variants thereof, are preferred over for example HeLA cells.

Alternatively, iPSCs can be generated for example from patient cells using methods known in the art, for example as described in U.S. Patent Application 20180023056 titled Reprogramming Method for Producing Induced Pluripotent Stem Cells (iPSC); U.S. Patent Application 20110306516 titled Methods for producing induced pluripotent stem cells and U.S. Pat. No. 8,048,999 titled Nuclear reprogramming factor and U.S. Pat. No. 9,580,689 titled Induced pluripotent stem cells, each of which are incorporated by reference in their entirety herein.

Effecting expression of Parkin polypeptide optionally wildtype or mutant labelled Parkin polypeptide, can for example be accomplished by culturing a cell under conditions suitable for protein expression, including for example culturing the cell at a growth permissive temperature, in a suitable culture medium, a sufficient time etc. that depend for example on the cell and desired expression level.

The expression may be effected by introducing into a cell a polynucleotide encoding Parkin or labelled Parkin, optionally wildtype or mutant labelled Parkin polypeptide, wherein the polynucleotides are operatively linked to one or more promoters and optionally comprised in one or more vectors.

The polynucleotide may be incorporated in a known manner into an appropriate expression vector, which ensures good expression of the polypeptide. Various constructs can be used. For example retroviral constructs such as lentiviral constructs are useful for expressing physiological levels of protein. Possible expression vectors include but are not limited to cosmids, plasmids, or modified viruses (e.g. replication defective retroviruses, adenoviruses and adeno-associated viruses), so long as the vector is compatible with the host cell used. The expression vectors are “suitable for transformation of a host cell”, which means that the expression vectors contain a nucleic acid molecule and regulatory sequences selected on the basis of the host cells to be used for expression, which is operatively linked to the nucleic acid molecule. Operatively linked is intended to mean that the nucleic acid is linked to regulatory sequences in a manner which allows expression of the nucleic acid.

Suitable regulatory sequences may be derived from a variety of sources, including bacterial, fungal, viral, mammalian, or insect genes (For example, see the regulatory sequences described in Goeddel, Gene Expression Technology: Methods in Enzymology 185, Academic Press, San Diego, Calif. (1990)). Selection of appropriate regulatory sequences is dependent on the host cell chosen as discussed below, and may be readily accomplished by one of ordinary skill in the art. Examples of such regulatory sequences include: a transcriptional promoter and enhancer or RNA polymerase binding sequence, a ribosomal binding sequence, including a translation initiation signal. Additionally, depending on the host cell chosen and the vector employed, other sequences, such as an origin of replication, additional DNA restriction sites, enhancers, and sequences conferring inducibility of transcription may be incorporated into the expression vector.

The recombinant expression vectors may also contain a selectable marker gene which facilitates the selection of host cells transformed or transfected with a recombinant molecule disclosed herein. Examples of selectable marker genes are genes encoding a protein such as G418 and hygromycin which confer resistance to certain drugs, β-galactosidase, chloramphenicol acetyltransferase, firefly luciferase, or an immunoglobulin or portion thereof such as the Fc portion of an immunoglobulin preferably IgG. Transcription of the selectable marker gene is monitored by changes in the concentration of the selectable marker protein such as β-galactosidase, chloramphenicol acetyltransferase, or firefly luciferase. If the selectable marker gene encodes a protein conferring antibiotic resistance such as neomycin resistance transformant cells can be selected with G418. Cells that have incorporated the selectable marker gene will survive, while the other cells die. This makes it possible to visualize and assay for expression of the recombinant expression vectors disclosed herein and in particular to determine the effect of a mutation on expression and phenotype. It will be appreciated that selectable markers can be introduced on a separate vector from the nucleic acid of interest.

Selection occurs in the absence of the metabolites e.g. glycine, hypoxanthine and thymidine for DHFR and glutamine for GS. Cells surviving selection comprise one or more copies of the transfected plasmid in the cell's genome. Further amplification of the copy number of the integrated DNA can be achieved by exposure of the selected cells to increasing levels of methotrexate (MTX) or methioninen sulphoximine (MSX) respectively. The recombinant expression vectors may also contain genes which encode a fusion moiety which provides increased expression of the recombinant protein; increased solubility of the recombinant protein; and aid in the purification of the target recombinant protein by acting as a ligand in affinity purification. For example, a proteolytic cleavage site may be added to the target recombinant protein to allow separation of the recombinant protein from the fusion moiety subsequent to purification of the fusion protein. Typical fusion expression vectors include pGEX (Amrad Corp., Melbourne, Australia), pMal (New England Biolabs, Beverly, Mass.) and pRIT5 (Pharmacia, Piscataway, N.J.) which fuse glutathione S-transferase (GST), maltose E binding protein, or protein A, respectively, to the recombinant protein.

Recombinant expression vectors can be introduced into host cells to produce a recombinant cell by one of many possible techniques known in the art. For example, a polynucleotide can be introduced by transforming a cell (e.g. electroporating a prokaryotic cell), transfecting a cell (e.g. using lipofectin) or transducing a cell (e.g. using a retrovirus). Prokaryotic cells can be transformed with a polynucleotide by, for example, electroporation or calcium-chloride mediated transformation. For example, polynucleotide can be introduced into mammalian cells via conventional techniques such as calcium phosphate or calcium chloride co-precipitation, DEAE-dextran mediated transfection, lipofectin, electroporation or microinjection. Suitable methods for transforming and transfecting host cells can be found in Sambrook et al. (Molecular Cloning: A Laboratory Manual, 3rd Edition, Cold Spring Harbor Laboratory Press, 2001), and other laboratory textbooks.

The mitophagy inducing agent may be selected from a proton ionophore, an iron chelator and a mitochondrial toxin.

In one embodiment, the proton ionophore is carbonyl cyanide m-chlorophenylhydrazone (CCCP). As shown in FIGS. 8 and 14, CCCP cause Parkin to undergo a subcellular transition from the cytosol to the mitochondria.

Various controls can be included. For example, the method can comprise comparing the amount of mitophagy induced by the MIA in the MIA contacted cells to the amount of mitophagy present in mitophagy vehicle control treated cells (e.g. in the absence of test agent). Further, the method can comprise contacting mitophagy vehicle control cells in one or more control wells with a mitophagy vehicle control to confirm the vehicle control does not substantially induce or prevent Parkin recruitment on its own.

The mitophagy vehicle control is typically the diluent used to dissolve the MIA. For example, the mitophagy vehicle control can be dimethyl sulfoxide (DMSO) when the MIA is CCCP diluted in DMSO.

In an embodiment, the labelled Parkin polypeptide label is a fluorescent label.

In one embodiment, the fluorescent label is selected from green fluorescent protein (GFP) including enhanced GFP and other variants, blue fluorescent protein (BFP) such as EBFP, EBFP2, Azurite, mKalama1, cyan fluorescent protein (CFP) such as ECFP, Cerulean, CyPet, mTurquoise2, and yellow fluorescent protein (YFP), such as YFP, Citrine, Venus, YPet.

In another embodiment, the labelled Parkin polypeptide label is a luciferase reporter enzyme.

In an embodiment, the Parkin polypeptide is detected directly or indirectly using an antibody conjugated to a label, optionally a fluorescent label, a light emitting label and the like or other label.

The mutant Parkin polypeptide, optionally the labelled Parkin polypeptide, can comprise one or more mutations, for example the mutation may be one or more of K161N, T240R, W403A and G430D, in the ring 0, ring 1 or ring 2 domain or any other mutation that impacts mitochondrial translocation.

In an embodiment, the method further comprises contacting wildtype control cells expressing for example labelled wildtype Parkin polypeptide comprising a wildtype Parkin polypeptide and a label in one or more wells with the test agent. Where the method is a high throughput assay, one or more of the plurality of wells are wildtype control wells that comprise cells expressing labelled wildtype Parkin. Such a step can for example provide mechanism of action information and can confirm any effect of the vehicle control.

In an embodiment, the control agent e.g the test agent vehicle control or the mitophagy vehicle control is selected from suitable vehicles for dissolving the TAs and/or MIAs (e.g. CCCP) such as water or DMSO.

In one embodiment, the test agent is assessed as a Parkin-mediated mitophagy inhibiting agent if the Parkin polypeptide experiences less than for example 50% translocation in the presence of a MIA, for example at least 10% less than a control treated with control agent and MIA. (e.g. the percentage of cells with mitochondrial Parkin polypeptide translocation in response to the test agent and CCCP is reduced by at least 10% compared to a control treated with control agent and CCCP).

In an embodiment, the mitophagy inducing agent is added to the cells in an amount that causes the endogenous or labelled wildtype Parkin polypeptide in control cells to experience greater than or about 60%, 70% or 80% translocation to mitochondria. For example, MIAs can be added to cause endogenous or labelled wildtype Parkin to experience between 60-80% translocation to mitochondria, including any percent between 60 and 80. This allows for inhibitors and activating agents to be determinable.

In one embodiment, the test agent is assessed as a Parkin-mediated mitophagy activating agent if the Parkin polypeptide experiences in test cells greater than 80%, 85%, or 90% translocation to mitochondria, for example at least a 10% increase compared to control cells.

As described in the Examples and herein, a Z score can be calculated, giving an indication of whether the test agent is an activating or inhibiting agent.

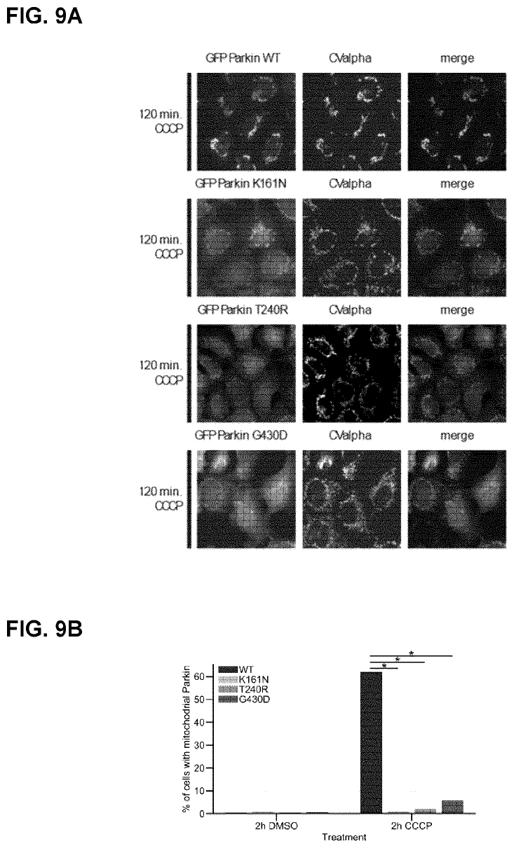

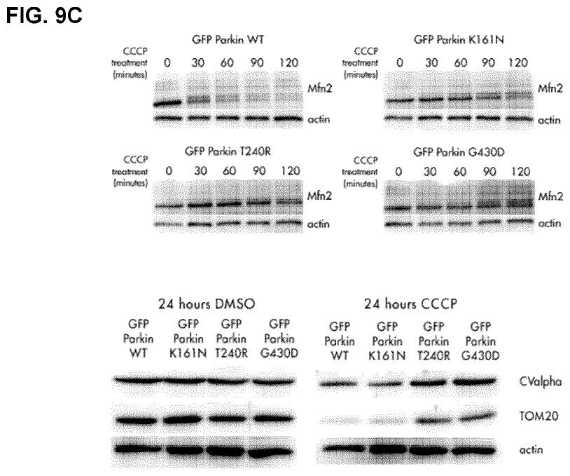

The amount of Parkin polypeptide, optionally labelled Parkin polypeptide (wild type and/or mutant), recruitment to mitochondria in the cells contacted with the test agent relative to the control cells can be measured as a percentage of cells comprising mitochondrial associated Parkin, for example as a percentage of cells with mitochondria in which the fluorescent label of the labelled Parkin polypeptide is detectable as abutting mitochondria, for example as shown in FIG. 9. This can also be assessed indirectly when Parkin is not labelled, using for example an immunocytochemical assay and detecting the label of a labelled antibody.

Accordingly, in an embodiment, the amount of Parkin polypeptide recruited is assessed by determining the amount of the cells in which Parkin polypeptide is recruited to the mitochondria relative to control cells.

The amount of Parkin polypeptide, optionally labeled Parkin polypeptide, recruited to mitochondria may be determined using a method that comprises detecting the labelled Parkin polypeptide, immunostaining the cells with a mitochondria detection agent, and identifying the proportion of labelled Parkin polypeptide that co-localizes with mitochondria detection agent, as shown in FIG. 9.

In an embodiment, the mitochondria detection agent is an anti-ATP5A antibody. In another embodiment, the mitochondria detection agent is an anti-TOMM20 antibody.

The methods may also include one or more positive controls. For example, it is demonstrated that SR3677 increases Parkin polypeptide recruitment. One or more wells can be treated with a Rho associated protein kinase (ROCK) inhibitor, as a positive control.

In some embodiments using labelled wildtype Parkin polypeptide, the amount of labelled wildtype Parkin polypeptide recruited to mitochondria in cells treated with test agent is compared to the amount of labelled mutant Parkin polypeptide recruited to mitochondria in cells treated with test agent.

In some embodiments using labelled wildtype Parkin polypeptide, the amount of labelled wildtype Parkin polypeptide recruited to mitochondria in cells treated with the control agent is compared to the amount of labelled mutant Parkin polypeptide recruited to mitochondria in cells treated with the control agent.

In an embodiment, the test agent determined to be a putative Parkin mediated mitophagy activating agent is further tested in one or more orthogonal assays for Parkin activity.

In one embodiment, the one or more assays is selected from assessing degradation of Mfn2, targeting of mitochondria to lysosomes and the clearance of mitochondria from cells.

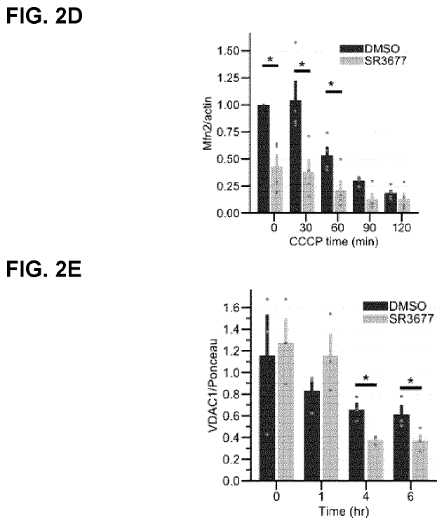

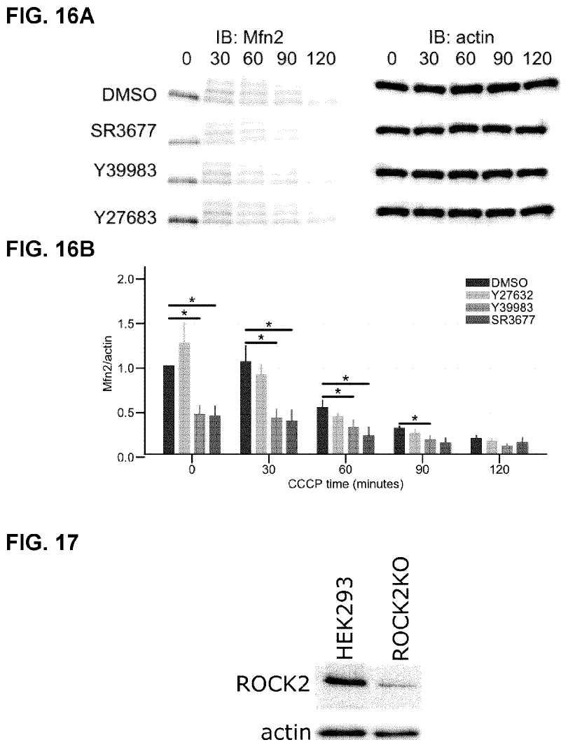

Degradation of Mfn2 may be detected, for example, by contacting cells expressing Parkin for example recombinantly labelled Parkin, with a test agent determined to be a Parkin mediated mitophagy activating agent, followed by contacting the cells and the control cells with CCCP, and detecting Mfn2 levels in the cells in relation to a control cell population. Mfn2 levels may be detected by western blot, as shown in FIG. 2B and FIG. 16.

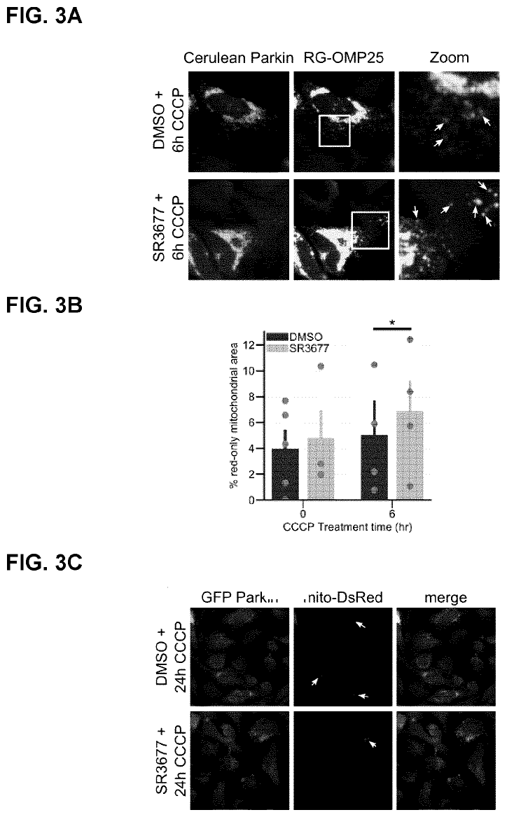

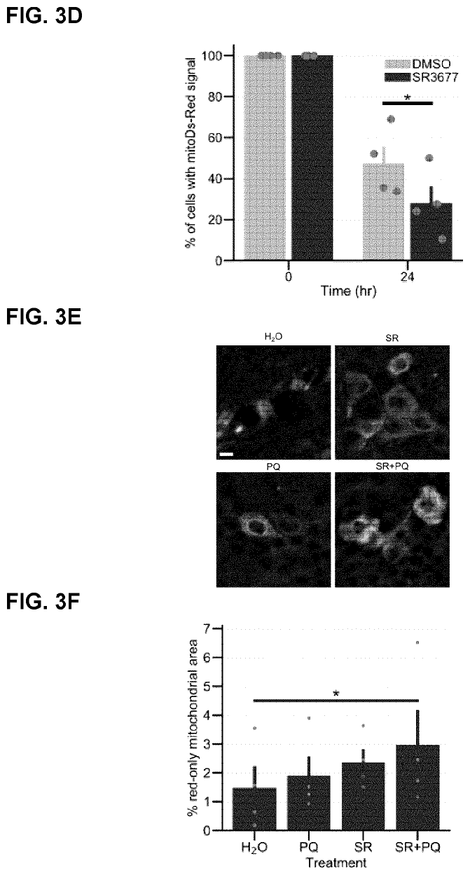

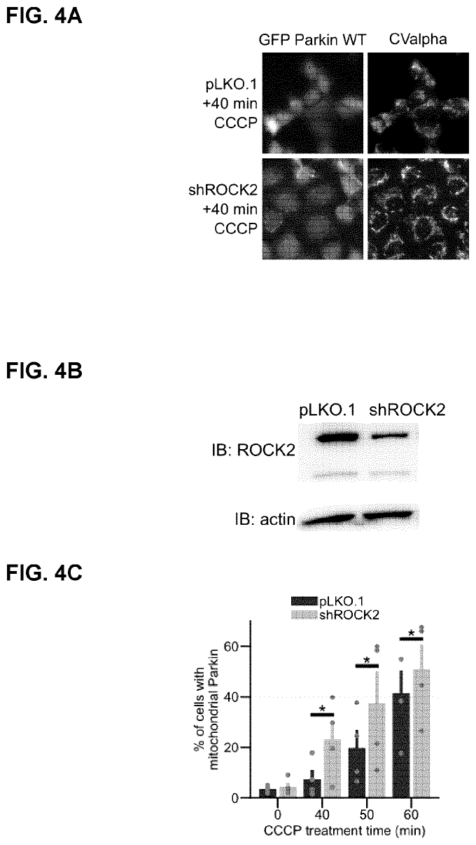

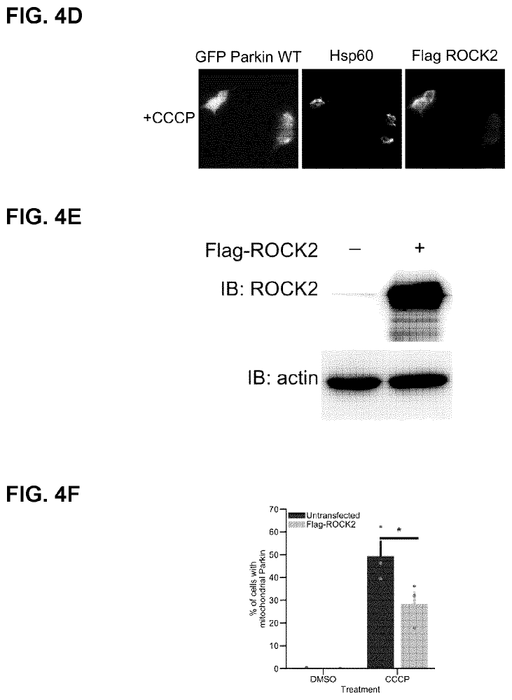

The mito-QC assay, may be used to detect the targeting of mitochondria to lysosomes. Cells expressing Parkin and a RGOMP25 plasmid may be contacted with a test agent determined to be a Parkin mediated mitophagy activating agent, followed by contacting the cells and the control cells with CCCP, and detecting the amount of GFP quenching in relation to a control cell population. Quenching may be detected by fluorescence microscopy as shown in FIG. 3A.

Clearance of mitochondria from cells may be detected by contacting cells expressing labelled mitochondria and GFP labelled Parkin with a test agent determined to be a Parkin mediated mitophagy activating agent and CCCP, where a decrease in mitochondria label over time in relation to control cells is indicative of mitochondria clearance. The amount of labelled mitochondria may be detected by fluorescence microscopy, as shown in FIG. 3C.

In an embodiment, the cells are contacted with the test agent for at least 30 min, 45 min, 60 min, 75 min, 90 min, 105 min or 120 min before contacting with the mitophagy inducing agent.

In an embodiment, the cells used to make the labelled Parkin polypeptide expressing cells are selected from HEK293 cells and SH-SY5Y cells. Any cells that express a suitable amount of endogenous level of wild type Parkin nucleic acid and/or polypeptide can be used. Suitable cells may include, for example cell lines MCF-7 and PC-3.

In an embodiment, the cells used are HEK293 cells. In another embodiment, the cells are induced-pluripotent stem cells (IPSCs), optionally having one or more mutations. For example, the iPSCs may be derived from patient fibroblasts having a mutation selected from G430D, T240R, W403A and K161N.

Any cells that express a suitable amount of endogenous wild type Parkin nucleic acid and/or polypeptide can be used. A suitable amount of endogenous Parkin nucleic acid and/or polypeptide is for example a comparable or greater amount than HEK293 or SH-Sy5Y cells and/or greater than the amount expressed by HeLa cells. The suitable level can be detected for example by immunofluorescence or western blot by comparing the level of expression in a cell type to HEK293 and/or HeLa cells.

In an embodiment, the test agent is added to the cells (e.g. well) at a final concentration of 0.4 μM to 40 μM.

In one embodiment, the test agent is added at a final concentration of 1 μM to 10 μM.

In a preferred embodiment, the test agent is added at a final concentration selected between from about 2 μM to about 4 μM.

In an embodiment, contacting the cells with test agent comprises pinning the test agent to the cells. In another embodiment, contacting the cells with test agent comprises pipetting the test agent on the cells.

In an embodiment, the method further comprises measuring the viability of the cells after the cells are contacted with the test agent, for example by staining the cells with a vital dye. For example, viability may be measured by staining cells with a crystal violet stain, and comparing level of crystal violet staining in wells exposed to different treatments.

The method can also include testing cell viability, for example using crystal violet or other viability dye, after for example addition of the test agent. If a test agent induces less than a selected threshold, for example 5% or less, of viability changes (e.g. cell death), the test agent is assessed as a Parkin-mediated mitophagy activating agent.

If a test agent induces more than twice the level of toxicity of the vehicle control (eg control agent), the test agent may be discarded as inducing toxicity.

In an embodiment, the amount of labelled Parkin polypeptide recruited to mitochondria in cells contacted with the test agent and the control cells is determined by:
i) segmenting the cell based on labelled Parkin polypeptide and a cell defining stain such as DAPI stain to identify individual cells, and
ii) classifying each cell of the cells according to whether the cell contains mitochondrial Parkin, which is assessed by determining if the cell comprises fluorescent Parkin signal abutting a signal of the mitochondrial detection agent, or lacks fluorescent Parkin signal abutting a signal of the mitochondrial detection agent.

Cells can be stained with a nuclear dye for example DAPI. Nuclear staining can aid for example in segmentation and classification.

The segmentation and classification of the cells may be performed using high content imaging (HCl) comprising automated microscopy, fluorescent detection and multi-parameter algorithms to visualize and quantify interactions in cell populations. Phenotypic changes such as morphology, cellular localization and proliferation can be monitored in high content screening (HSC) for hit identification or in high content analysis (HCA).

Optionally, the segmentation and classification of the cells is performed using CellProfiler Analyst software or PhenoLOGIC machine learning software.

In one embodiment, the segmentation and classification of the cells is performed using artificial intelligence computer software that analyzes 2 or more, optionally 3 areas of cell images and compares the images to images of cells defined as having Parkin localized to the mitochondria or cytosol.

The Cells may be split into 2 sub-populations: cells with even labelled Parkin distribution and cells where labelled Parkin is localized to mitochondria.

In one embodiment, at least 200, at least 250 or at least 300 cells are assessed per treatment group or well.

The test agents may be rank ordered based on z-score, which may be calculated, for example, using the formula:

$$z = \frac{x - μ}{σ} ,$$

where x is the percentage of cells with mitochondrial GFP-Parkin for a specific small molecule, μ is the average percentage of cells with mitochondrial Parkin in positive control columns where cells are treated with CCCP only.

In an embodiment, the amount of labelled Parkin polypeptide recruited to mitochondria in the cells contacted with the test agent and the control cells is determined by image acquisition software. For example, cells may be first imaged using a microscope configured with a camera capable of detecting fluorescence. Image acquisition may performed digitally using image acquisition software configured to work the camera. Capture images may then be segmented using software such as CellProfiler Analyst.

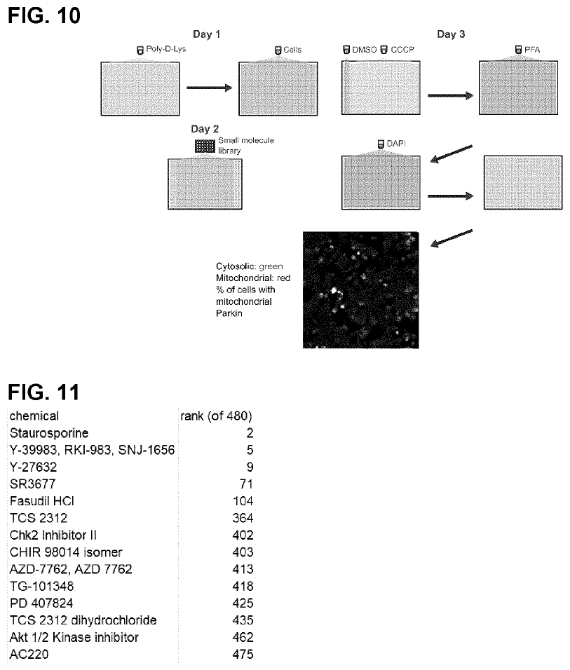

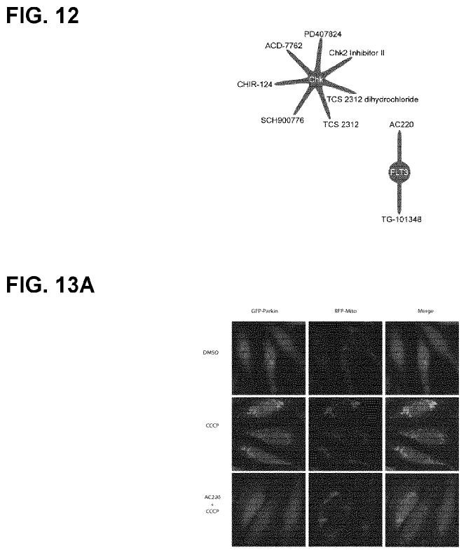

In an embodiment, the cells are grown on a cell-adherence agent coated surface prior to contacting the cells with the test agent, as shown in FIG. 10. The cell-adherence agent may be or comprise, for example, poly-lysine.

In an embodiment, the cells are allowed to adhere to the cell-adherence coated surface for 24 hours prior to contacting with the test agent. The cells can be plated for example at 80% confluency, or between 70% and 90% confluency.

In an embodiment, the test agent is a molecule from a small molecule library. The small molecular library may be, for example, a kinase inhibitor library.

Hits can be assessed in one or more other assays. For example, hits can be evaluated for chemical similarity. For example, SMILEs can be obtained for activator compounds using the Python library PubchemPy (https://pubchempy.readthedocs.io), which provides programmatic access to the Pubchem compound database (https://pubchem.ncbi.nlm.nih.gov/). SMILEs can be converted into Morgan fingerprints using the Python Library RDKit⁴⁴. Principle component analysis can be conducted to reduce the dimensionality of the data, using the Python library scikit-learn (http://scikit-learn.org/stable/). This allows for evaluation of chemical similarity based on the proximity of their corresponding data points in 2D space.

Hits can also be assessed to confirm that they are not apoptosis inducing.

For example, Pubmed literature can be mined to eliminate hits documented to induce apoptosis and/or to remove hits that on their own damage mitochondria and result in Parkin recruitment. This can also be tested in a further assay such as an apoptotic assay, optionally Annexin 5 staining.

In an embodiment, the method is configured for performance in a high-throughput assay.

In another aspect the present disclosure provides a high-throughput screening assay comprising:
preparing a plurality of test wells and control wells each comprising cells expressing endogenous Parkin polypeptide comprising a wildtype or a mutant Parkin polypeptide sequence,
contacting the plurality of test wells with a plurality of test agents
contacting the plurality of test wells with either a mitophagy inducing agent (MIA) or a mitophagy vehicle control, wherein one or more of the plurality of test wells are contacted with the MIA or the mitophagy vehicle control,
measuring an amount of Parkin polypeptide recruited to mitochondria in the plurality of test wells,
comparing the amount of Parkin polypeptide recruited to mitochondria in the plurality of test wells contacted with TA to the amount of Parkin polypeptide recruited to mitochondria in the plurality of test wells contacted with the MIA and optionally a control agent, and
comparing the amount of Parkin polypeptide recruited to mitochondria in the plurality of test wells contacted with MIA to the amount of labelled Parkin polypeptide recruited to mitochondria in the plurality of test wells contacted with the mitophagy vehicle control.

In some embodiments, the methods further comprise determining a Z score, for example as described herein. The method can for example be used to categorize hits. Hits with a high z score may be selected, optionally for validation in a further assay, for example an assay described herein.

In another aspect the present disclosure provides a high-throughput screening assay comprising:
preparing a plurality of test wells and control wells each comprising cells recombinantly expressing labelled Parkin polypeptide comprising a wildtype or a mutant Parkin polypeptide sequence and a label,
contacting the plurality of test wells with a plurality of test agents
contacting the plurality of test wells with either a mitophagy inducing agent (MIA) or a mitophagy vehicle control, wherein one or more of the plurality of test wells are contacted with the MIA or the mitophagy vehicle control,
measuring an amount of labelled Parkin polypeptide recruited to mitochondria in the plurality of test wells,
comparing the amount of labelled Parkin polypeptide recruited to mitochondria in the plurality of test wells contacted with TA to the amount of labelled Parkin polypeptide recruited to mitochondria in the plurality of test wells contacted with the control agent, and
comparing the amount of labelled Parkin polypeptide recruited to mitochondria in the plurality of test wells contacted with MIA to the amount of labelled Parkin polypeptide recruited to mitochondria in the plurality of test wells contacted with the mitophagy vehicle control.

This high-throughput screening method provides for replicates of increasing amounts to assess for linear relationships.

In another embodiment, wherein the Parkin polypeptide is endogenous Parkin localization is measured using an immunological method, optionally using a labelled primary anti-Parkin antibody or using a secondary labelled antibody that detects the primary anti-Parkin antibody. The antibody can be conjugated to a fluorescent tag or other label, which is measured to indicate the localization of Parkin and for example to identify which cells have mitochondrial Parkin.

As mentioned above, typically, the assay will employ at least labelled Parkin comprising a mutant Parkin sequence. A Parkin polypeptide sequence that is wild type can, in some embodiments be used alone, if for example the method is for identifying test agents that increase wild type Parkin recruitment. In other embodiments, the assay will involve expressing mutant Parkin and wildtype Parkin.

In an embodiment, the high-throughput screening assay uses cells expressing endogenous Parkin. For example, the assay can also use iPSCs derived from a patient having a Parkin mutation. In such methods, where the Parkin is not labelled, anti-Parkin antibodies are used to detect the endogenous Parkin. In other embodiments, the endogenous Parkin is wild-type. For example, in embodiments, using mutant Parkin, molecules that restore mutant function are identified. In embodiments, using wildtype Parkin, molecules that agonize normal wild type Parkin are identified.

Techniques for high throughput cellular screening assays are known to those skilled in the art. Briefly, the workflow shown in FIG. 10 may be performed in microplates including a grid of wells typically in multiples of 96. Microplates may range in size from 96 wells to 384 wells, to over 1000 wells. Using liquid handling components, compounds may be added to the wells in an automated fashion. Liquid handling components can include robotic components such as plate handlers for the positioning of microplates, automated lid or cap handlers to remove or replace lids, tip assemblies for sample distribution with disposable tips, washable tip assemblies, microplate loading blocks, reagent racks, microtiter plate stacking towers, and computer systems.

The methods can also comprise one or more steps described in the Examples. In an embodiment, the method comprises one or more steps or is as described in FIG. 10.

### III. Kits

In another aspect, the present disclosure provides a kit comprising one or more of
a vector such as a plasmid, comprising a mutant Parkin expression cassette for expressing labelled Parkin polypeptide comprising mutant Parkin polypeptide sequence and a label; and/or
cells expressing wildtype or mutant Parkin polypeptide, optionally recombinant cells comprising a mutant Parkin expression cassette for expressing labelled Parkin polypeptide comprising mutant Parkin polypeptide sequence and a label and one or more of
a multi-well plate, including a multi-well plate described herein,
a pintool head for delivering small volumes,
a vector such as a plasmid, comprising a wildtype Parkin expression cassette for expressing labelled wildtype Parkin polypeptide,
recombinant cells comprising a wildtype Parkin expression cassette for expressing labelled wildtype Parkin polypeptide, and
one or more transfection reagents.

Any one of the components or a combination of the components, can be in a package optionally, separated in vials or other containers.

The following non-limiting Example is illustrative of the present disclosure:

### EXAMPLES

### Example 1

### Stable Cell Line Development

GFP Parkin plasmids were transfected into HEK293 cells using Lipofectamine2000 (Invitrogen, 11668027) according to manufacturer's instructions. Cells stably expressing GFP Parkin were selected using 800 ug/mL geneticin (Gibco, 11811031). FACS sorting was performed to select for cell populations expressing GFP at similar levels.

HEK293 GFP Parkin cells were transfected with shRNA targeting ROCK2 or with pKO1 control vector (Sigma, SHC001). Puromycin (Biobasic, PJ593) selection was performed to select for cells that have stably incorporated this construct. ROCK2 KO cell lines were generated using clustered regularly interspaced short palindromic repeats (CRISPR)/Cas9 gene editing. Briefly, gRNA targeting sites were designed within the first exon of ROCK2. Two oligonucleotides containing the target sequences were annealed and cloned into the PX458 vector (Addgene plasmid #48138). Following transfection of this construct or the parental PX458 vector into HEK293 cells, single GFP-positive cells were sorted into 96-well plates for colony isolation.

### Small Molecule Screening

10 uL of 0.1 mg/mL Poly-D-Lys solution was added to 384-well plates for 5 minutes, followed by a PBS wash. Plates were dried for at least 2 hours. 50 μL of 600 000 HEK293 GFP Parkin cell/mL of DMEM was dispensed into each well. After 24 hours incubation to allow the cells to adhere, 200 nL of small molecules (or DMSO in columns 1, 2, 23 and 24) were pinned. Following a 16-hour incubation, 200 nL of 5 mM CCCP were added to all wells except columns 1 and 2, into which 200 nL of DMSO was added. Following 2-hour incubation, cells were fixed with 4% paraformaldehyde (PFA) and stained with 50 μL of 1 μg/mL DAPI solution for 15 minutes. After the final PBS wash, plates were ready for high content microscopy.

Images were acquired on IN Cell Analyzer 6000 (GE Healthcare), equipped with sCMOS camera (2048×2048), and 20x/0.45 NA Plan Fluor objective (Nikon) in open aperture mode using 1×1 binning. Image analysis was performed using Columbus Image Analysis System (PerkinElmer). Nuclei were initially detected in DAPI channel, followed by whole cell segmentation in GFP channel. Using PhenoLOGIC machine learning plug-in, cells were split into 2 sub-populations: cells with even GFP-Parkin distribution and cells where GFP-Parkin was localized to mitochondria. The percentage of cells with mitochondrial GFP-Parkin was determined per well. The molecules were then rank ordered based on z-score, which is calculated using the formula:

$$z = \frac{x - μ}{σ} ,$$

where x is the percentage of cells with mitochondrial GFP-Parkin for a specific small molecule, μ is the average percentage of cells with mitochondrial Parkin in positive control columns 23 and 24, where cells are treated with CCCP only.

### Chemicals

The following chemicals were used in the study: CCCP (Sigma, C2759), SR3677 (Tocris, 3667/10), Y27632 (Millipore, 688000), Y39983 (MedChemExpress, HY-10069), paraquat (Sigma, 36541), E-64 (BioShop, EEL640.1), leupeptin (BioShop, Leu001.50), geneticin (Gibco, 11811023), puromycin (BioShop, PUR333.10), retinoic acid (Calbiochem, 554720), brain-derived neurotrophic factor (Gibco, PHC7074), Chloroquine (Bioshop, CHL919).

Constructs, shRNA and Antibodies

Cerulean-Parkin, RG-OMP25 have previously been described^{7,8}. R777-E285 Hs.ROCK2 (Addgene plasmid #70569). The Gateway recombination system (Life Technologies) was used to insert ROCK2 into pDEST-pcDNA5-BirA-FLAG, which has been previously described¹⁰. BirA was removed by inverse PCR.

ShRNA against ROCK2 was purchased (Sigma, TRCN0000184636).

The gRNA target oligonucleotides were phosphorylated with T4 polynucleotide kinase (NEB, M0201S) and annealed in a thermocycler: and inserted into the PX458 vector (Addgene plasmid #48138).

The following primary antibodies were used in the study: mouse anti-ATP5A (Abcam, 14748), rabbit anti-Hsp60 (Abcam, 46798), rabbit anti-ROCK2 (Abcam, 125025), mouse anti-actin (Abcam, 8226), anti-HK2 (Abcam, 3740910), mouse anti-Mfn2 (Abcam, 56889), anti-Tom20 (Santa Cruz, 390545), anti-VDAC1 (Abcam 14734) anti-UQCRC2 (Abcam 14745), rabbit anti-COXIV (Novus, NB110-39115) and mouse anti-Flag (Sigma, F1804).

Rabbit and mouse horseradish peroxidase-conjugated secondary antibodies (Jackson Immunoresearch) were used as secondary antibodies.

### Cell Culture

Cells were cultured in Dulbecco's modified Eagle's Medium (DMEM) supplemented with 10% fetal bovine serum (Sigma, F1051) at 37° C. in humidified air containing 5% CO₂. Cells were tested for mycoplasma contamination (Lonza, LT07).

### Immunoblotting

Lysates were harvested using lysis buffer (0.1M Tris HCl, 0.01% SDS, pH 9) with 1× protease inhibitor cocktail (BioShop, PIC002.1). Lysates were then heated at 95° C. for 20 minutes with vortexing performed at 5-minute intervals. The BCA assay (Pierce, 23227) was performed to determine protein concentration, to standardize protein loading across samples to be compared. 12% SDS-PAGE gels were used to run samples to be analyzed for LC3 conversation and for Tom20 and 10% gels were run for all other samples. Transfer of proteins onto PVDF membrane (Immobilon, IPVH00010) was performed at 110V for 80 minutes or at 36V for 8 hours. Membranes were blocked with 5% skim milk in TBST (1×TBS, 0.1% Tween-20 [BioShop, 1M23298]) for 30 minutes prior to incubation in primary antibodies at a dilution of 1:1000 for all antibodies except anti-ROCK2 and anti-LC3, which were used at a dilution of 1:5000. Visualization of proteins was performed using ECL (BioRad, 11705062).

Phostag western blotting was carried out using the same protocol, with one exception: following electrophoresis, the gel was washed 3 times with transfer buffer containing 10 mM EDTA for 10-minute intervals and one final time with only transfer buffer prior to protein transfer.

Densitometry was performed by normalizing bands corresponding to the protein of interest to a loading control such as actin using ImageLab 6.0 software (BioRad).

### Immunofluorescence

Cells on coverslips were washed with PBS, incubated in 4% paraformaldehyde for 15 minutes, washed again with PBS prior to incubation in 0.1% Triton X-100 for 15 minutes. After a final PBS wash, cells were then incubated in 10% goat serum (Gibco, 16210072) for 30 minutes. Cells were then incubated in primary antibodies diluted in PBS at 1:500 concentration, or at concentration specified by the manufacturer at 4° C. overnight. Following a PBS wash, coverslips were incubated with Alexa Fluor secondary antibodies (Life Technologies, A-11001, A-11004 and A-11011) at a concentration of 1:500. Coverslips were then mounted onto slides using Fluoromount-G (Invitrogen, 00495802).

### Mito-QC Assay

HeLa cells were seeded into 6-well plates containing glass coverslips and allowed to adhere overnight. Cells were co-transfected with RGOMP25 and Cerulean-Parkin plasmids 24 hours prior to treatments. E-64 and leupeptin were added along with either DMSO or 10 μM CCCP for 6 hours prior followed by a PBS wash, fixation with 4% PFA and a final PBS wash prior to mounting onto glass slides with Fluoromount.

Cells were treated with 50 μM chloroquine for 16 hours prior to addition of E64, leupeptin, SR3677 and CCCP in the indicated treatment group. For the in vivo mitoQC assays, 5 7-day old male TH-GAL4>UAS-mitoQC were placed into vials containing water (control), sublethal 1 mM paraquat, 1 mM SR3677 alone and in combination with paraquat for 7 days. Whole flies were then fixed overnight in 1% PFA and 0.1% Tx-100. Fly brains were then dissected and fixed with 4% PFA for 20 minutes. 3 10-minute PBS washes were performed in between each step. Fly brains were mounted in VectaShield™ mounting medium for imaging using the Zeiss LSM700 confocal fluorescence microscope with 40× 1.4 NA Oil Plan-APOCRAMAT objective and the appropriate lasers and filter. 0.8 μm z-stacks were acquired to capture dopaminergic neurons expressing the mitoQC transgene. Laser settings were kept constant within each trial. At least 2 fly brains were imaged for each treatment across 4 independent trials.

Quantification of the RG assay was done using Fiji software. The perimeter of the cell was selected by drawing a region of interest (ROI) around cells that have been transfected with Cerulean-Parkin and RGOMP23. A red/green fluorescence intensity ratio was determined for each pixel. Pixels were considered red-only if the red/green fluorescence ratio was greater than or equal to 1.5. The area of red-only pixels over the total non-background area was determined and averaged across 4 independent trials in which at least 20 cells were quantified per treatment.

### Mitochondrial Clearance Assay

HeLa cells stably expressing GFP parkin and mito-DsRed were seeded into 12-well plates containing coverslips and allowed to adhere overnight. Cells were then treated with DMSO or 0.5 μM SR3677 for 2 hours prior to addition of 10 μM CCCP for 24 hours. Cells were then washed with PBS, fixed with 4% PFA and washed with PBS again prior to the mounting of coverslips onto glass slides. Mitochondrial clearance was quantified by counting the number of cells in each treatment to retain mito-DsRed signal. 3 independent trials were performed with at least 50 cells quantified per trial.

### Parkin Recruitment Assay

HEK293 GFP Parkin cells were seeded into 12-well plates containing coverslips. Cells were treated with small molecules for 2 hours prior to mitophagy induction with 10 μM CCCP, unless indicated otherwise. Following fixation with 4% PFA, immunostaining was carried out using anti-ATP5A primary antibody and anti-mouse Alex Fluor 568.

To measure the percentage of cells with Parkin localized to mitochondria a 2 step process was employed: (1) whole cell segmentation based on GFP Parkin using CellProfiler software and (2) classification into Parkin distribution subpopulation using CellProfiler Analyst. The percentage of cells with Parkin localized to mitochondria was determined in each image. Images were taken at 20× magnification for this experiment, resulting in the classification of at least 300 cells in each treatment group per trial.

### Mitochondrial Isolation

2 confluent wells of a 6-well plate were fractionated into total, cytosolic and mitochondrial fractions using the Mitochondrial Isolation Kit for Cultured Cells (Abcam, 110170).

### Cell Viability Determination

SH-SY5Y cells were differentiated according to previously established protocol consisting of sequential treatment with retinoic acid followed by brain-derived neurotrophic factor¹⁰. Cells were then seeded into white 96-well plates with white bottom and allowed to adhere overnight. Varying doses of SR3677 were combined with 500 μM paraquat and administered for 24 hours. Controls treated with 0.5 μM SR3677, equivalent volume DMSO and paraquat alone were also included. Following 24-hour incubation, cell media was aspirated and THE lysis buffer (50 mM Tris-HCl, 100 mM NaCl, 0.1 mM EDTA) containing protease inhibitor cocktail was added to each well. Half of the lysate volume was removed and aliquoted into a separate 96-well plate. ATP levels were measured in the opaque plate using the ATP determination (ThermoFisher A22066) assay kit and protein concentrations were measured using the Pierce BCA (ThermoFisher #23225) assay kit. ATP levels were normalized to protein concentration and then by the ratio for cells treated with paraquat alone in a control well on each plate.

### Drosophila Stocks

All stocks were maintained at 25° C. and at 70% relative humidity in 12 h light/dark cycles and were fed standard yeast-molasses-agar medium. The following fly lines were obtained from Bloomington Drosophila Stock Center: UAS-park^{RNAi} (31259), UAS-rok^{RNAi} (34324), ddc-GAL4 (8848), tubulin-GAL4 and Canton (S).

### Drosophila Longevity and Climbing Assays

Male Canton(S) Drosophila were aged 7 days prior to feeding with standard fly food supplemented with water, 10 mM paraquat, 1 mM SR3677, or with combinations of these chemicals. Flies were placed into new vials containing fresh food supplemented with the treatments previously administered and 2 days later, climbing assays were performed. Flies were placed into plastic cylinder vials and tapped down, so all flies fell to the bottom. The number of flies to climb beyond 12.5 cm was counted every 10 s and values are represented as the percentage of total flies in each treatment group.

### Statistical Analysis

Paired student's t tests were used for all analyses, unless otherwise specified. Logistic Regression was performed to construct dose response curves, which was used to determine EC₅₀ values. Survival analyses were performed using the log-rank test to analyze whether a difference in the survival rates between treatments was significant. At least 3 independent biological replicates were performed for all experiments for which statistical analysis was performed. All figure legend sample sizes (n) refer to the number of independent experiments performed. P-values below 0.05 were considered significant.

### Chemical Similarity Determination

SMILEs for all activator compounds were obtained using the Python library PubchemPy (https://pubchempy.readthedocs.io), which provides programmatic access to the Pubchem compound database (https://pubchem.ncbi.nlm.nih.gov/). SMILEs were converted into a Morgan fingerprints using the Python Library RDKit⁴⁴. Principle component analysis was conducted to reduce the dimensionality of the data, using the Python library scikit-learn (http://scikit-learn.org/stable/). This allows for evaluation of chemical similarity based on the proximity of their corresponding data points in 2D space.

### Protein Target Similarity Determination

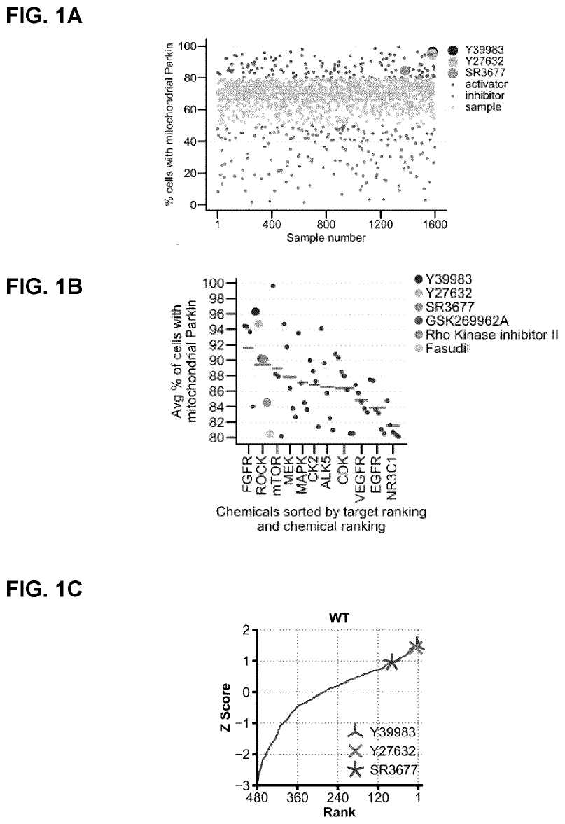

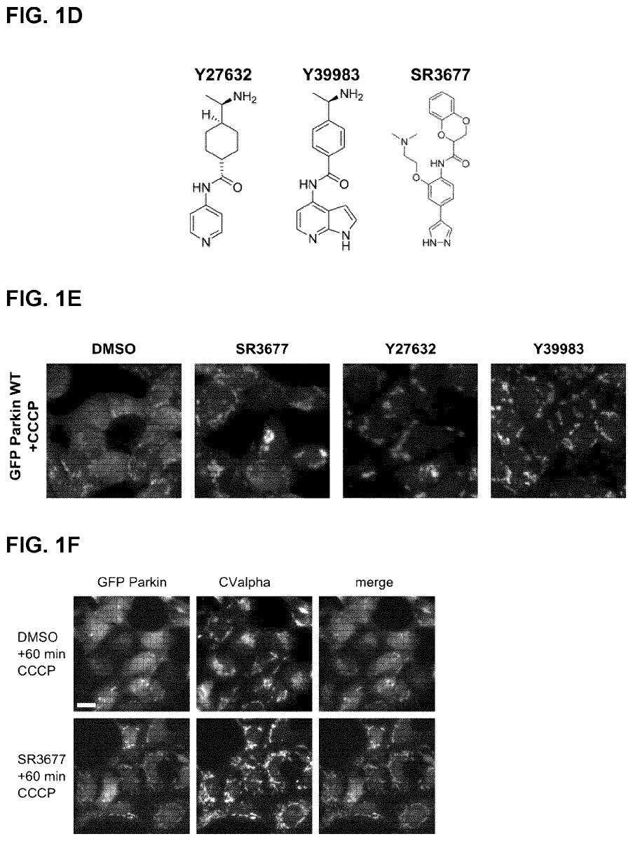

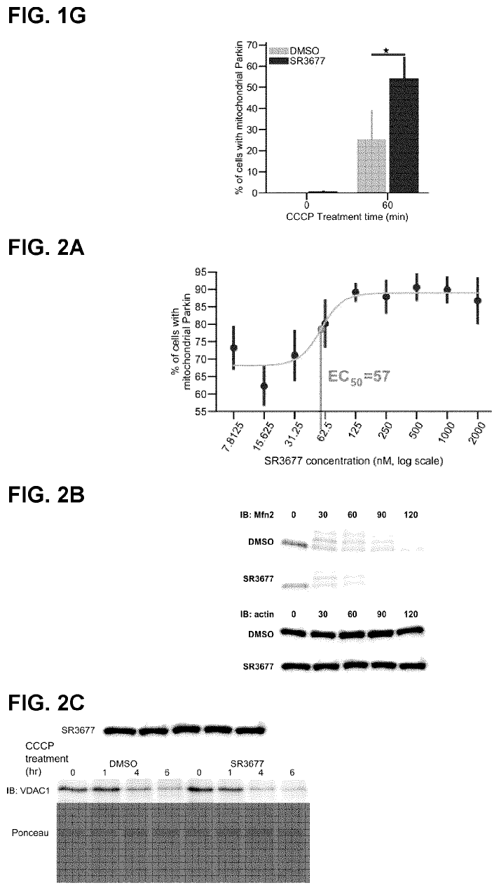

Protein target information was provided by the chemical library supplier or acquired from Drugbank database (https://www.drugbank.ca/). Activators were grouped based on common targets and average activity values (% of cells with mitochondrial Parkin) were determined for each family of compounds with a common target. Screening leads were prioritized for validation according to the average activity values and the number of molecules belonging to each family of activators (FIG. 1).

### Results

A high throughput small molecule screen aimed at identifying molecules that increase the proportion of cells with Parkin localized to mitochondria upon induction of mitophagy with the protonophore, carbonyl cyanide m-chlorophenylhydrazone (CCCP) (FIG. 8) was developed. This subcellular transition is impaired in several PD-linked Parkin mutants, resulting in the impaired clearance of outer mitochondrial membrane (OMM) substrates (FIG. 9) and the reduced turnover of damaged mitochondria (FIGS. 9A, B)^{11,12}.

HEK293 cell lines stably expressing GFP-tagged wild-type Parkin, as well as 3 Parkin mutants with missense mutations localized to 3 distinct domains critical for Parkin's function which fail to localize to damaged mitochondria as rapidly as wild-type Parkin were created and screened. These cell lines were screened across 2 biological replicates with compounds from the kinase inhibitor (n=480) and Prestwick (n=1120) collections. The 3-day screening protocol consisted of (1) seeding cells, (2) pinning small molecules at 4 μM concentration and (3) inducing mitophagy with CCCP, followed by processing of plates for high content imaging. Following image acquisition, DAPI staining was used to segment cells within each image. Supervised machine learning was used to classify cells into two classes: (1) cells with cytosolic Parkin distribution and (2) those with Parkin localized to mitochondria. The % of cells with mitochondrial Parkin in wells pinned with DMSO, instead of small molecule, upon CCCP addition was 83.4±2.3% and 71.2±2.6% for the kinase inhibitor and Prestwick library screens, respectively. The average % of cells with mitochondrial Parkin in negative control wells, untreated with CCCP were 3.7±2.6% and 1.1±1.1% for the kinase inhibitor and Prestwick library screens, indicating good separation between negative and positive controls. Compounds which increased the proportion of class 2 cells across 2 biological replicates, compared to the CCCP-only treated cells (FIG. 10), as indicated by their z-score (FIG. 11), were considered hits.

The percentage of cells with mitochondrial Parkin distribution in the negative (DMSO alone) and positive (CCCP alone) control wells were generally set at <10 and >60%, respectively. Staurosporine, a known activator of Parkin-mediated mitophagy^{13,14}, was recovered in the top 0.3% of hits, indicating the robustness of the pipeline to identify true Parkin-mediated mitophagy activators.

Small molecules which yielded average values greater than 80% were considered candidate hits, while those yielding average values below 50% were considered inhibitors (FIG. 1a). To maximize odds of recovering true positives³⁴, Parkin recruitment activators were arranged according to their protein target (FIG. 1b). Inhibitors of FGFR, ROCK and MEK had the highest average Parkin recruitment values. Of these three, the ROCK inhibitor series was the largest recovered, with 6 ROCK inhibitors identified as activators. The average percentage of cells with mitochondrial Parkin amongst these 6 compounds is 89.4%, in the kinase inhibitor screen where the positive controls average 71.2%. Another compelling reason to follow up on this family of inhibitors is the evidence in the literature for autophagy enhancement and improvement of phenotypes associated with neurodegeneration following treatment with these compounds^{6,17}

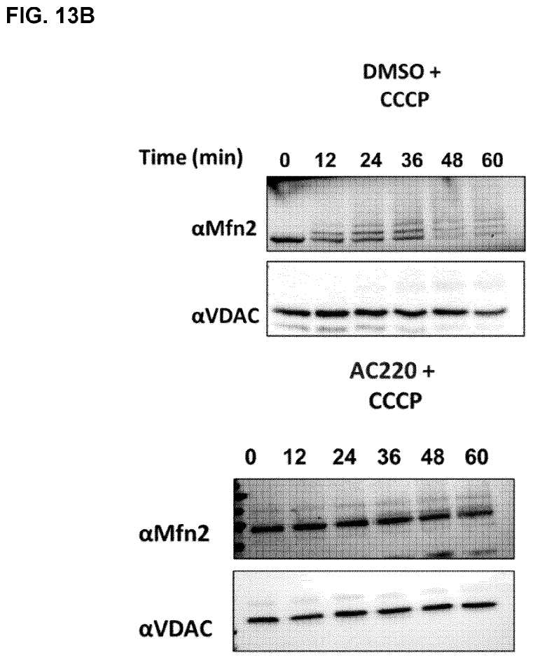

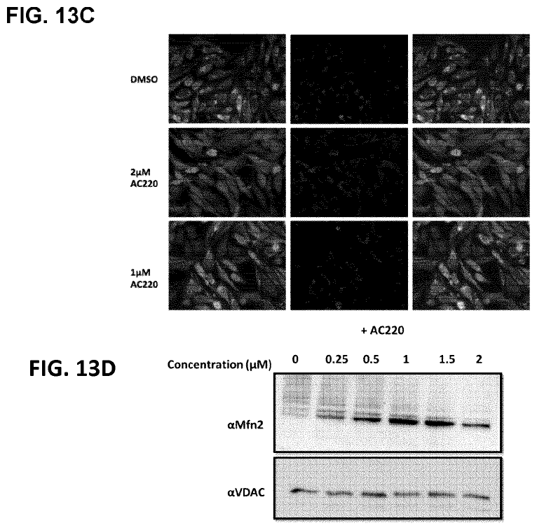

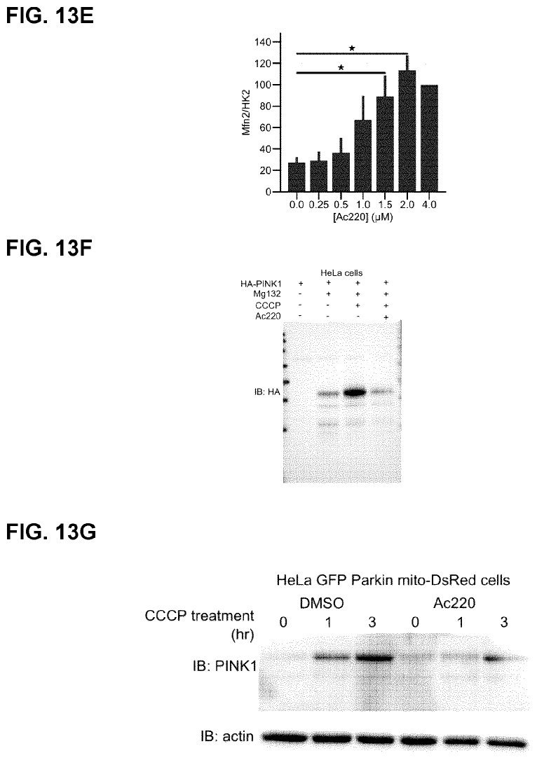

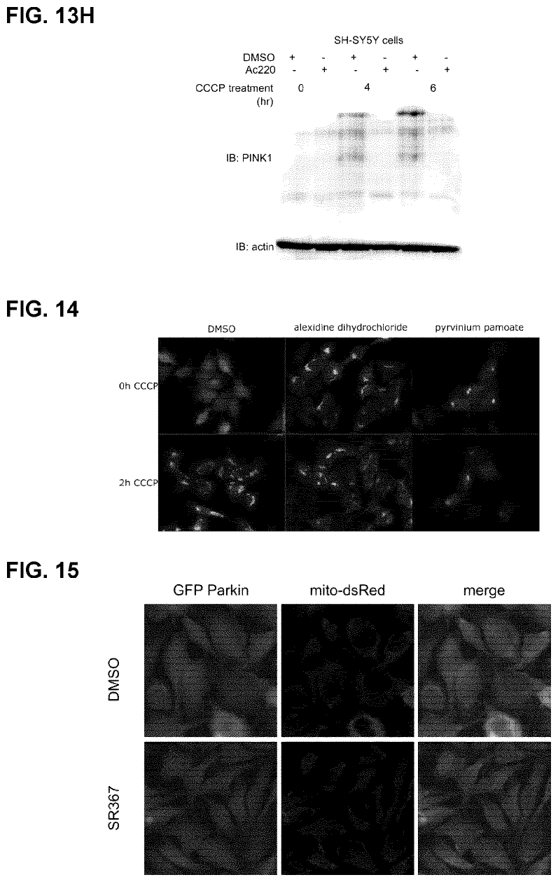

An isolated analysis of the kinase inhibitor data revealed small clusters of molecules with the same effect on Parkin recruitment, that interestingly also have a common canonical target. Several Chk inhibitors were identified as inhibitors of Parkin localization to damaged mitochondria (PD 407824, 425/480; Chk2 inhibitor II, 402/480; TCS 2312 dihydrochloride, 435/480; TCS 2312, 364/480; CHIR-124, 403/480; AZD-7762, 413/480). Two inhibitors of FLT3 (Ac220, 475/480; TG-101348, 418/480) were identified as mitophagy inhibitors as well. Further experiments confirmed the inhibitory effect of Ac220 on Parkin recruitment following mitochondrial damage (FIG. 13).

Notably, identification of mitophagy-activating compounds is fraught by the enrichment of hits whose mechanism of action involves induction of apoptosis by mitochondrial damage. To account for this, the Pubmed literature was mined to eliminate any hits documented to have this effect and to also remove hits that on their own damage mitochondria and result in Parkin recruitment.

Several inhibitors of Rho-associated protein kinase (ROCK) were identified as top hits in the kinase inhibitor library screen (Y39983, 5/480; Y27632, 9/480; SR3677, 71/480; FIG. 1). Y27632 and Y39983 are used as biological probes in numerous studies. These compounds sufficiently inhibit both ROCK1 and ROCK2 in both biochemical and cell-based assays (for a review, see Feng et al., J. Med. Chem. 2016¹⁵), however due to poor cell permeability are administered at high concentrations in cell-based assays ranging from 10-100 μM^{16,17.} SR3677 selectively inhibits ROCK2, the isoform predominantly expressed in CNS tissue, at nM concentration, according to previous studies¹⁸. Surprisingly, SR3677 ranked lower than the other ROCK inhibitors in the kinase inhibitor library screen despite the increased potency, perhaps the result of toxicity issues at the dose (4 μM) which were screened. Therefore, various doses of SR3677 were tested in the Parkin recruitment assay to determine the optimal concentration to use in follow up experiments (FIG. 2A). Principle component analysis was performed on Morgan fingerprints corresponding to each activator to evaluate chemical similarity amongst the hits³⁴. Besides the common protein target of these Parkin recruitment activators, the 3 hits selected for further validation share the greatest degree of structural similarities (FIG. 23).

Surprisingly, SR3677 ranked lower than the other ROCK inhibitors in our kinase inhibitor library screen despite its lower IC₅₀ in cell-based assays¹⁵. Thus, the effect of varying doses of SR3677 on Parkin recruitment was re-tested to determine the optimal concentration to use in subsequent experiments (FIG. 1c). By 0.5 μM, the maximal effect of SR3677 was achieved and increased Parkin recruitment to damaged mitochondria was observed following long (17 hour) and short (2 hour) pre-incubation (FIG. 1). Importantly, Parkin distribution was diffuse in cells treated with SR3677 in the absence of CCCP. Parkin recruitment activators which cause mitochondrial damage (FIG. 14)^{35,36}, recruit Parkin in the absence of CCCP.

SR3677 at 0.5 μM concentration was chosen in subsequent experiments to ensure induction of a maximal response. The amino-pyrimidine series of ROCK inhibitors (Y27632 and Y39983) were re-tested at 10 μM concentration, as described in previous studies. The compounds were re-tested after 2-hour treatment, and recapitulated the effects observed following overnight incubation (16 hours). Importantly, Parkin distribution was diffuse when cells were treated with the compounds alone (FIG. 15).

In addition to enhancers of Parkin recruitment to damaged mitochondria, the screen identified several inhibitors of Parkin recruitment to damaged mitochondria. By elucidating the mechanism of action of these molecules, previously uncharacterized regulators of the Parkin-mediated mitophagy pathway may be identified which may guide the development of therapeutics in the future. Parkin-mediated mitophagy inhibitors may be tested in PD model systems which exhibit excessive mitophagy, such as PD-causing mutations W403A in PARK2 and A53T in PARK1³⁷.

The Parkin recruitment inhibitor families identified in our screen include compounds targeting FLT3, EGFR, MET, CDK, JAK, checkpoint (CHK) and Aurora (AURK) kinases, in addition to prostaglandin synthase (PTGS) and TUB bipartite transcription factor (FIG. 24). Both Aurora (AURK) and cyclin-dependent (CDK) kinases promote Drp1 activity and its mitochondrial recruitment, which are both prerequisites for stabilization of PINK1 on the outer mitochondrial membrane^{46,38}.

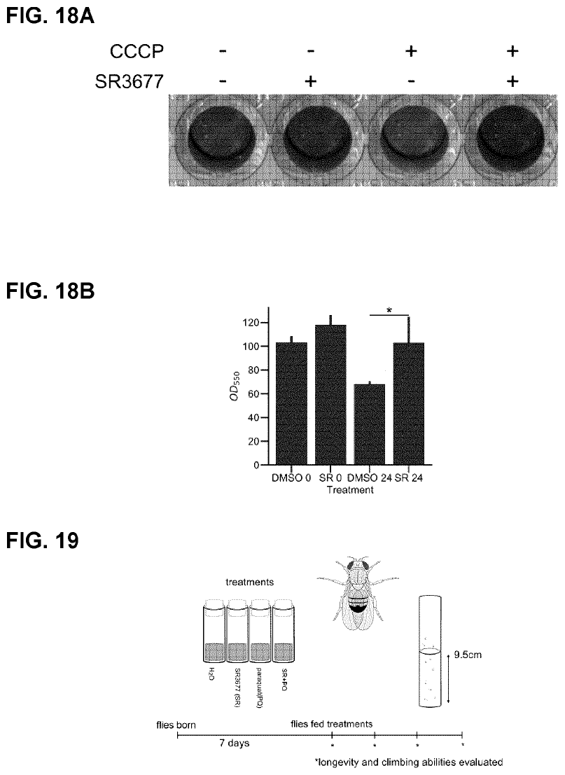

One group of Parkin recruitment inhibitors identified were FLT3 inhibitors, such as Ac220 (FIG. 24). Upon retesting, Ac220 inhibited Parkin recruitment and degradation of one of its outer mitochondrial membrane substrates, Mfn2 (FIGS. 13, 18)²⁰. Mfn2 degradation is critical for driving mitophagy forward by facilitating the segregation of damaged mitochondria from the healthy mitochondrial network and the dissociation between the ER and the mitochondria^{21,22} Following Ac220 treatment, PINK1 fails to accumulate in response to mitochondrial damage (FIG. 13).

Parkin recruitment and Mfn2 degradation were inhibited by Ac220 in a dose-dependent manner (FIG. 13). Currently, the most frequently employed methods to inhibit mitophagy consist of blocking lysosomal acidification (bafilomycin or chloroquine) or general autophagy (PI3K inhibitors)¹⁴. Specific inhibitors of this pathway may serve as valuable chemical tools.

Mfn2 is rapidly ubiquitinated and degraded following induction of mitochondrial damage^{19,18}, and this event is thought to be critical for driving mitophagy forward. Mfn2 rapid degradation promotes the segregation of damaged mitochondria from the healthy mitochondrial network and also facilitates the dissociation of the ER from mitochondria^{21,22}. Mfn2 degradation was assessed in the screening cell line following CCCP treatment. Increased degradation of Mfn2 was observed following treatment with Y39983 and SR3677 (FIG. 2B), however the greatest effect was achieved following SR3677 treatment (FIG. 2B, FIG. 16). In contrast to Ac220, ⅔ ROCK inhibitors (Y39983, SR3677) enhanced Mfn2 degradation (FIG. 2). However, in order to achieve comparable increases to Mfn2 degradation, Y39983 had to be administered at 10 μM, while SR3677 could significantly enhance Mfn2 degradation at 0.5 μM, a 20-fold lower concentration. The poor cell permeability of the amino-pyridine series of ROCK inhibitors likely accounts for this difference in working concentration.

Consistently, SR3677 increased the turnover of another outer mitochondrial membrane Parkin substrate, VDAC1^{20,39} (FIG. 2). The degradation of proteins residing in other submitochondrial compartments was also examined. SR3677 increased the degradation of inner mitochondrial membrane proteins ATP5A and COXIV. Likewise, the degradation of UQCRC2, a matrix-facing subunit of the IMM protein COXIII, was enhanced by SR3677 co-treatment. Both UQCRC2 and ATP5A are represented within Parkin's ubiquitylome³⁹.

Mitochondrial mass in HeLa cells expressing GFP-Parkin and mito-DsRed were also quantified, where DsRed is targeted to the mitochondrial matrix. Previous studies have found that prolonged depolarization leads to complete loss of mito-DsRed signal in a large proportion of cells⁸, so the percentage of cells which retained mito-DsRed signal to assess mito-DsRed clearance was quantified. While both DMSO- and SR3677-treated cells retained mitochondrial mass following 24 hour DMSO treatment, 47.5±8.25% of cells pre-treated with DMSO retained mito-DsRed following 24 hour CCCP treatment, compared to only 28±8.21% of cells pre-treated with SR3677 (FIG. 3). These orthogonal methods show that SR3677 enhances degradation of proteins in various submitochondrial compartments.

Next, the effect of SR3677 on downstream steps in the Parkin-mediated cascade was assessed. The mito-QC assay was used to evaluate targeting of damaged mitochondria to lysosomes. This method exploits the differential pH-sensitivity of the mCherry and GFP fluorophores, to distinguish intact mitochondria from those localized to lysosomes. Upon localization within the acidic environment of the lysosome, GFP is more rapidly quenched so red-only signal corresponds to lysosome-localized mitochondria. mCherry and GFP are expressed alongside the transmembrane domain of the OMM protein, OMP25. Pre-treatment of cells with SR3677, increased the extent of mitochondrial targeting to lysosomes (FIG. 3A, B). This only occurred when mitophagy was induced with CCCP. To confirm that the SR3677-mediated increase to the percentage of red-only mitochondrial area is dependent on autophagy, the effect of SR3677 in cells pre-treated with chloroquine was tested. Chloroquine inhibits the fusion of autophagosomes and lysosomes⁴⁰. It was observed that pre-treatment with chloroquine abrogates SR3677-mediated enhancement of mitophagy.

Next, the clearance of mitochondria was examined following pre-treatment with SR3677. Prolonged treatment with CCCP induces depolarization of the entire mitochondrial network and leads to the complete clearance of mitochondria from Parkin-expressing cells^{8,12}. The percentage of cells retaining mitochondrial signal decreases in cells pre-treated with DMSO prior to CCCP administration. This reduction is enhanced further when cells are instead pre-treated with SR3677. The findings indicate that SR3677 increases Parkin-mediated mitophagy at several sequential steps in the pathway leading to an overall enhancement of mitophagy.

Increased Parkin recruitment and Mfn2 degradation (FIG. 1, FIG. 16) was observed, following administration of ROCK inhibitors from two distinct chemical families: the amidopyridine and the amidophenylpyrazole series. The distinct effects of these compounds in kinase panel screens for selectivity suggested that these compounds are likely acting through their canonical target, rather than a common off-target. Nevertheless, whether genetic manipulation of ROCK2 would phenocopy the effect of SR3677 was investigated. Since the hypomorphic phenotype produced by shRNA more closely mimics the effect of a small molecule inhibitor, this method was used in the mitophagy assays and CRISPR-mediated genome editing was used for antibody validation (FIG. 17)²³.

After creating and validating HEK293 GFP Parkin cell lines stably expressing shRNA against ROCK2, Parkin recruitment was examined. Increased recruitment of Parkin was observed following mitophagy induction in cells expressing shROCK2, compared to the pKO1 control vector. While reduction of ROCK2 levels phenocopied SR3677 administration, overexpression of Flag-ROCK2 reduced the proportion of cells with Parkin localized to mitochondria following CCCP treatment. The similar effect of SR3677 and ROCK2 knockdown on Parkin recruitment and the opposing effect of ROCK2 overexpression indicate that ROCK2 negatively regulates the Parkin-mediated mitophagy pathway and validates target engagement in vivo.

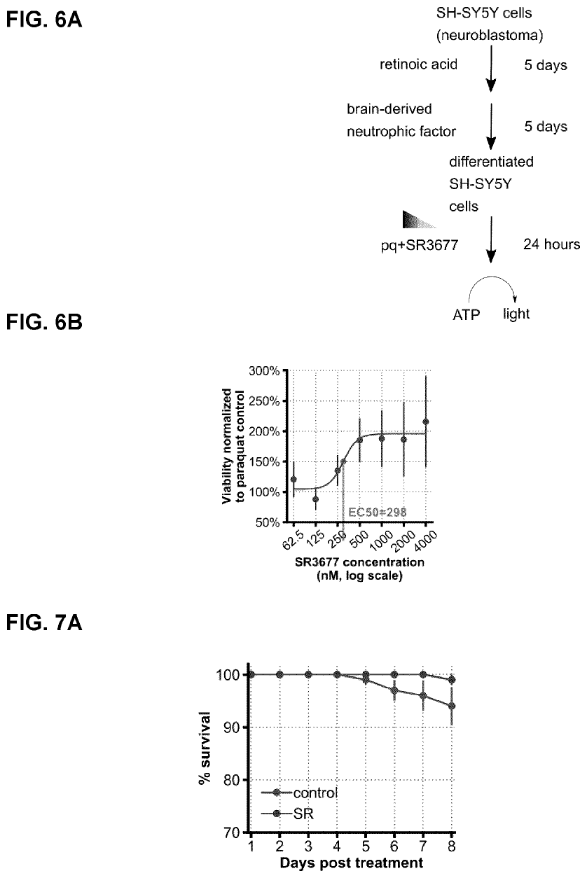

To determine whether these findings could be translated into dopaminergic neurons, the study was continued in the dopaminergic neuroblastoma SH-SY5Y cell line. Parkin undergoes a similar CCCP-dependent subcellular transition from cytosol to mitochondria in these cells¹². First, whether the viability of these cells would be improved by SR3677 was investigated. Initially, experiments were conducted using crystal violet staining as an indicator of cell viability. Prolonged CCCP treatment (24 hours) reduced the amount of staining, indicating the likely detachment of dead cells. Pre-treatment of cells with SR3677 increased crystal violet, as indicated by measuring OD₅₀₀ (FIG. 18). Next, SH-SY5Y cells were differentiated into a more neuron-like population according to protocols previously described¹⁰ and ATP levels were measured. Paraquat treatment alone reduced the viability of cells, while 0.5 μM SR3677 treatment did not affect cell viability. Varying concentrations of SR3677 were co-administered alongside the PD-causing toxin paraquat. Notably, SR3677 improved the viability of cells challenged with paraquat in a dose-dependent manner (FIG. 6b).

An increase Parkin recruitment was observed following administration of 6 different ROCK inhibitors (FIG. 1). Several of the ROCK inhibitors diverge significantly with respect to their structures, suggesting that these activators likely function by binding ROCK, rather than through a common off-target interaction (FIG. 23). Additionally, the specificity of SR3677 has been demonstrated in kinase panel screens¹⁵.

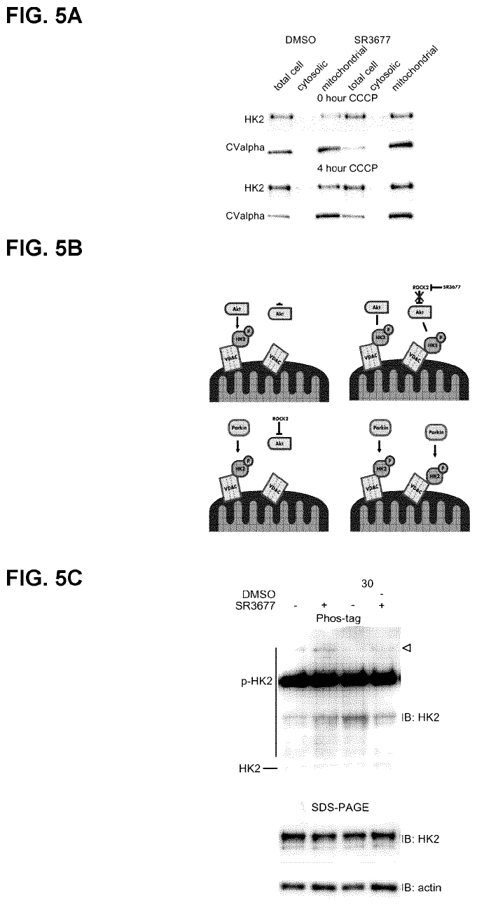

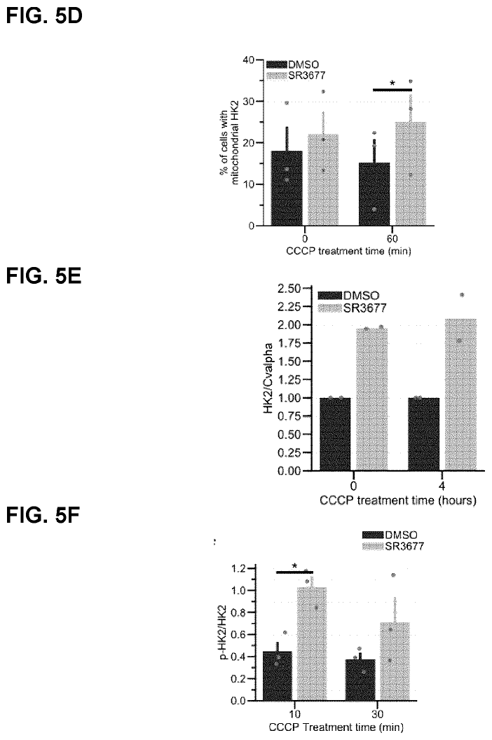

To gain mechanistic insight into how ROCK inhibition upregulates Parkin recruitment to damaged mitochondria, the pathways that are downstream were examined. ROCK activates PTEN, a negative regulator of Akt. The requirement for Akt-mediated activation of HK2 has been observed in an RNAi-based screen for Parkin recruitment modulators, and furthermore in subsequent experiments conducted in the SH-SY5Y cell line. Akt-mediated phosphorylation activates HK2 and promotes its translocation to the mitochondria. To determine whether HK2 may be involved in the SR3677-mediated effect on Parkin-mediated mitophagy, SH-SY5Y cells were fractionated into total cell, cytosolic and mitochondrial fractions following treatment with either DMSO or SR3677. More HK2 was observed in the mitochondrial fraction following treatment with SR3677 (FIG. 5a), suggesting that the effect of the ROCK inhibitors on Parkin-mediated mitophagy are mediated through this pathway. Phostag gels were run to assess the phosphorylation status of HK2 following treatment with either DMSO or SR3677. Increased phosphorylation of HK2 was observed following treatment with ROCK inhibitor (FIG. 5), further substantiating this possible mechanism of action.

Increased phosphorylation of HK2 was observed following ROCK inhibitor treatment (FIG. 5). Since HK2 is also a Parkin substrate, HeLa cells which lack endogenous Parkin were used to examine the effect of SR3677 treatment on HK2 distribution⁴³. SR3677 treatment increases the mitochondrial distribution of HK2 following mitochondrial damage (FIG. 5a, b). Together these results show that SR3677 increases the activity and mitochondrial localization of a positive regulator of Parkin recruitment, HK2.

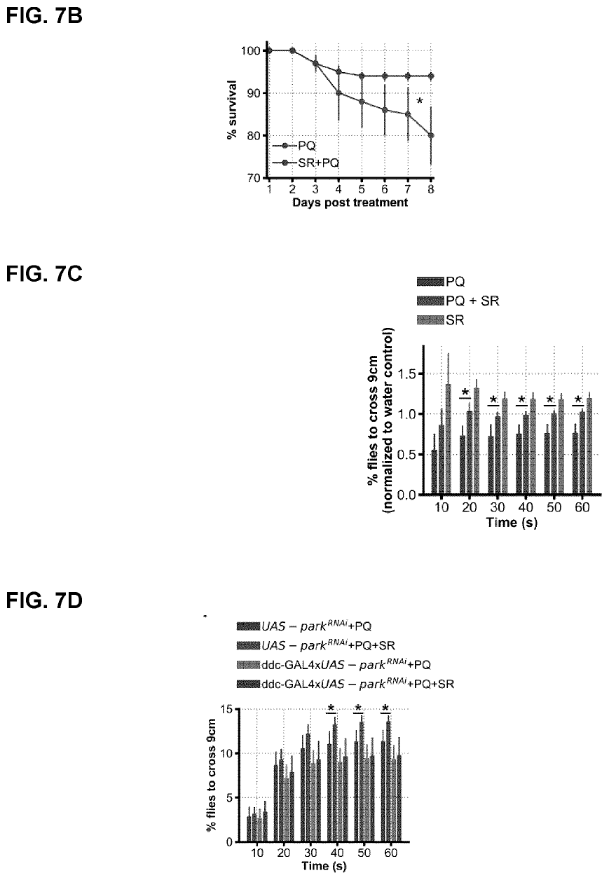

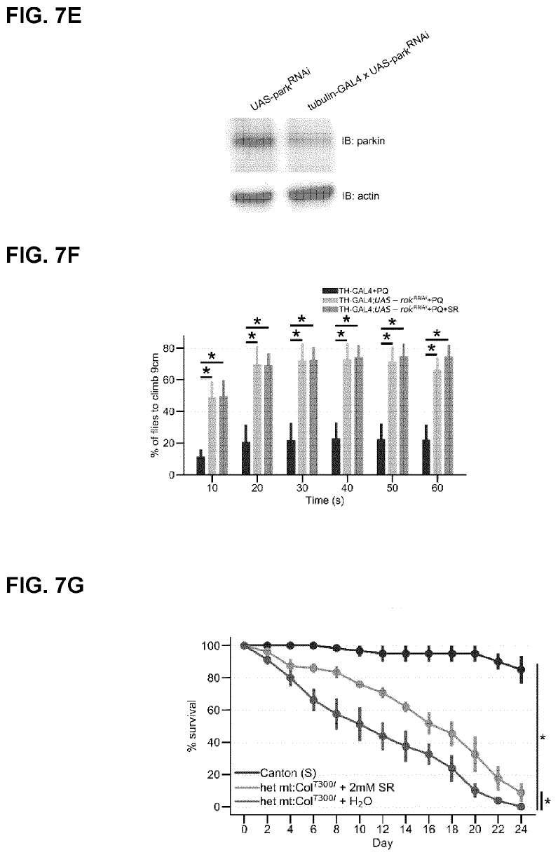

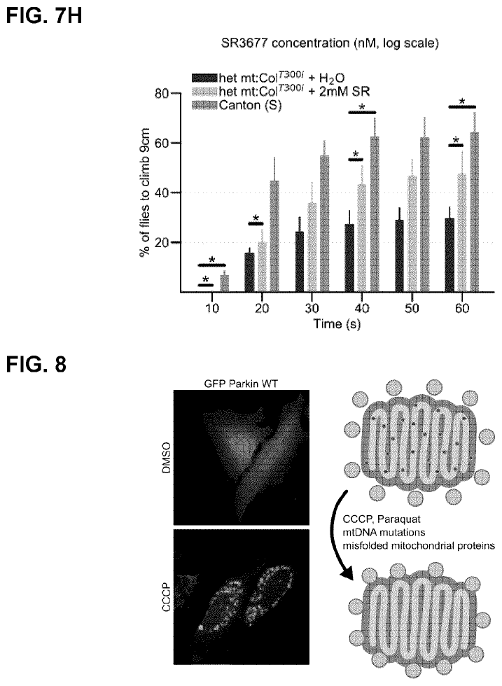

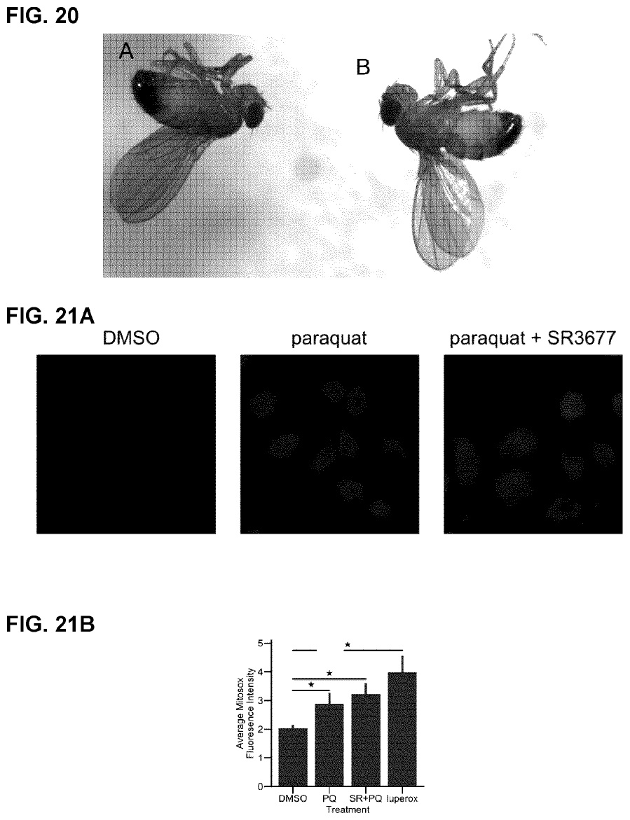

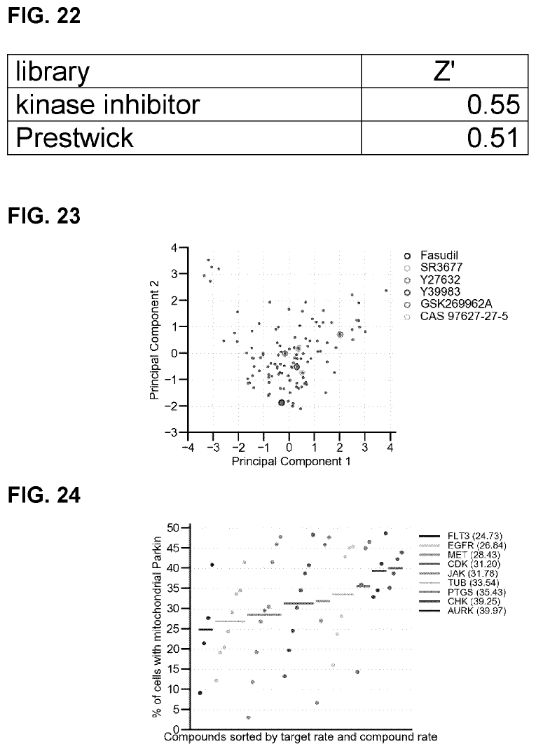

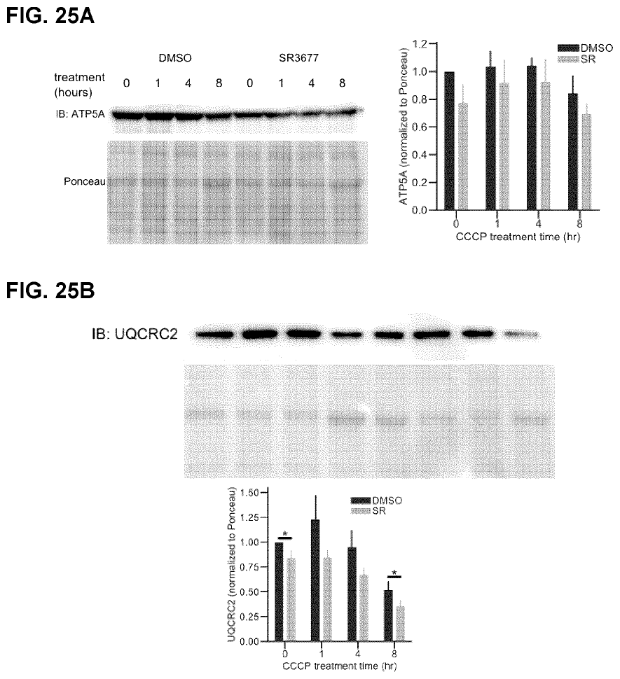

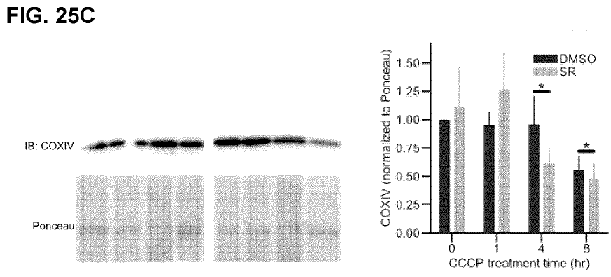

Next, whether the effects observed in neurons may translate when tested in a more complex PD model, Drosophila, was investigated. Flies with mutations in genes encoding PINK1 and Parkin display reduced longevity and reduced locomotor function^{24,25}. Flies fed paraquat display similar phenotypes. This neurotoxin model was used to test the effect of SR3677 on PD-related phenotypes. The amino acid sequences of Drosophila Rho-associated kinase (Drok) were aligned to ROCK2, the human isoform with which it is most similar. The amino acids predicted to be essential for binding of SR3677 to human ROCK2 (Met-172, Glu-170, Lys-121, Asp-176) were conserved in the Drok sequence. Briefly, 7-day old Canton(S) male flies were fed a standard diet supplemented with various combinations of paraquat and SR3677 (FIG. 19). The survival of flies was reduced following administration of paraquat, as previously reported²⁶⁻²⁸. Co-administration of SR3677 significantly improved viability of flies challenged with paraquat (FIG. 7A, B). Feeding SR3677 alone does not affect survival of flies. Paraquat administration impairs the climbing ability of flies over time (FIG. 7C). Notably, co-treatment with SR3677 restores climbing ability in paraquat-treated flies. To verify that flies were consuming paraquat and SR3677, the fly food and drugs were supplemented with blue food dye and blue coloring in the abdomens of the flies was observed (FIG. 20).

The mitoQC assay may also be performed in Drosophila to quantify mitophagy specifically in cell types of interest using the GAL4/UAS system. Briefly, the mitoQC transgene was expressed in dopaminergic neurons using the TH-GAL4 driver⁴¹. Since CCCP cannot be administered without affecting the viability of the flies, 7-day old flies were fed the parkinsonian toxin, paraquat. Paraquat has been used to induce mitochondrial dysfunction and to model PD in Drosophila. In addition, paraquat is a known inducer of Parkin recruitment⁴². Following 7-days of treatment, fly brains were dissected and imaged. The mitoQC transgene was expressed in dopaminergic neuron clusters in the fly brain. Red dots corresponding to mitochondria localized to lysosomes were evident in all treatments examined, to varying extents. The percentage of red-only mitochondrial signal over the total mitochondrial signal in flies co-treated with SR3677 and a sublethal paraquat dose (1 mM) was greater than flies whose food was supplemented with water (FIG. 3).

Whether SR3677 may protect against paraquat-induced phenotypes by interfering with paraquat's activity was also assessed. Paraquat gives rise to mitochondrial superoxide species that can be detected using mitoSOX, a fluorescent probe that is targeted to mitochondria, where it may be oxidized by superoxides. The oxidized mitoSOX species fluoresces upon encountering nucleic acid. The fluorescence intensity of Schneider's S2-R⁺ cells, a cell line derived from dissociated embryos with a flat morphology amenable to imaging, was assessed following treatments with paraquat or paraquat and SR3677. Treatment with both paraquat and luperox, a tert-butyl hydroperoxide that induces oxidative damage, increases the fluorescence intensity of mitoSOX within the cells. Co-treatment with SR3677 does prevent the paraquat-mediated increase in mitochondrial superoxide levels, indicating that SR3677 likely does not interfere with paraquat activity (FIG. 21) and is thereby working in vivo by engaging its target to promote mitophagy.

Since previous studies have described the neuroprotective effect of ROCK inhibition in other PD models, such as in rodents challenged with the PD neurotoxin MPTP^{29,30}. Other pathways modulated by ROCK were attributed for these effects, or other Akt activation alone. To test whether Parkin-mediated mitophagy pathway may be required for the neuroprotective effects observed following ROCK inhibitor treatment, dopamine decarboxylase (ddc)-GAL4 flies were crossed with transgenic UAS-park^{RNAi} flies to specifically knockdown Parkin in the dopaminergic neurons of the fly. These flies and the parental UAS-park^{RNAi} fly line were fed vehicle control (water) or SR3677 along with paraquat. The climbing ability of the UAS-park^{RNAi} flies was improved when SR3677 was co-administered alongside paraquat. SR3677 co-administration produced no improvement to paraquat-induced climbing defects in ddc-GAL4>UAS-park^{RNAi}. It was also observed that ddc-GAL4>UAS-rok^{RNAi} flies fed paraquat displayed better climbing ability than paraquat-fed UAS-rok^{RNAi} flies. No further improvement to climbing ability was observed in TH-GAL4; UAS-rok^{RNAi} flies fed SR3677 alongside paraquat (FIG. 7) indicating the requirement for Parkin.

Finally, SR3677 in an alternate, genetic model of mitochondrial dysfunction was tested. Specifically, flies with a temperature-sensitive, de-stabilizing mutation in cytochrome c oxidase subunit I (mt:Col^{T300l}) were used. This mutation causes depolarization of the mitochondrial membrane potential and increased mitochondrial reactive oxygen species⁴⁴, manifesting in systemic consequences such as impaired climbing and survival. Flies with this mutation in 100% of their mitochondrial genomes, or homoplasmic flies, only survive 4 days post-eclosion. In order to extend our therapeutic window, SR3677 in heteroplasmic flies which contain this temperature-sensitive mutation in approximately 90% of their mtDNA instead⁴⁷ was tested. These flies retain the same phenotypes as homoplasmic flies, including reduced survival and climbing ability although to a less severe extent (FIG. 7).

Immediately following eclosion, male heteroplasmic mt:Col^{T300l} flies were transferred to vials containing either water, 1 or 2 mM SR3677. Newly eclosed Canton(S) male flies were placed into fresh vials and referenced as a control for these experiments and flies were raised at non-permissive 29° C. for these experiments. Survival was equally improved by 1 mM and 2 mM supplementation of SR3677 into fly food. Climbing ability was also improved by 2 mM SR3677 addition. In agreement with the effect of SR3677 co-administration on paraquat-mediated survival and climbing deficits (FIG. 7), this model replicates the improvement to phenotypes arising from mitochondrial dysfunction.

Taken together, these data demonstrate the efficacy of inhibiting ROCK as a neuroprotective strategy.

### DISCUSSION

In this study, it was found that ROCK levels and activity contribute to the regulation of Parkin-mediated mitophagy. Numerous downstream substrates are activated by ROCK, including PTEN, a negative regulator of Akt. McCoy et al., demonstrated the requirement for Akt-mediated activation of HK2 for the recruitment of Parkin to damaged mitochondria. Likewise, they demonstrated that over-expression of mediates increased Parkin recruitment. The same effect was exerted by impinging on the negative regulators of HK2 pharmacologically. After validating the upregulation of Parkin-mediated mitophagy at several steps including the recruitment of Parkin to damaged mitochondria, the clearance of mitochondrial substrates and the targeting of mitochondria to lysosomes, the significance of these effects in vivo were tested. Differentiated SH-SY5Y cells and Drosophila demonstrated improved survival when challenged with the PD-causing toxin paraquat. While previous studies have demonstrated the neuroprotective effect of ROCK inhibition in PD models, this study established that these effects in the model were dependent on the presence of Parkin in the dopaminergic neurons. This is the first study to connect the neuroprotective effect of ROCK inhibition to Parkin-mediated mitophagy upregulation, despite the usage of mitochondrial toxins in many of these studies^{31,32}. By gaining insight into the mechanism of action of these promising small molecules, it will be possible to apply them most effectively, including to rationally design combinatorial therapies.

Interestingly, in Japan a ROCK inhibitor called Fasudil and its derivative have been used for the treatment of vasospasm following subarachnoid hemorrhage. These compounds have demonstrated safety and efficacy in this application. Furthermore, by using ROCK2-specific inhibitors it may be possible to deliver these effects in the brain tissue where they are required. One drawback to note, is the poor bioavailability of SR3677. Further optimization will be required before proceeding to test this molecule in relevant preclinical model organisms, such as non-human primates. Alternatively, it may be possible to use the analog SR3850, which was not represented in the screen, but which demonstrates improved pharmacokinetics properties³³.

Future attempts to increase the pool of Parkin localized to damaged mitochondria may benefit from two strategies: (1) to activate HK2 directly or (2) to inhibit negative regulators of the Akt-HK2 axis. The first strategy may employ a similar method of structure-guided design of neo-substrates, as has been employed to successfully identify PINK1 activators. For the second strategy, inhibitors of negative regulators of Akt, such as PTEN, may be screened for their ability to potentiate Parkin. Currently, the most potent and selective inhibitor of PTEN identified is SF1670. While the screening efforts of this study included approximately 3000 molecules, PTEN inhibitors were not represented in the data set. These compounds can be tested alone and in combination with the ROCK inhibitors identified in this study, given that they may have an additive effect by impinging on the same pathway. The increased capacity of dopaminergic neurons to eliminate damaged mitochondria may lead to the development of much-needed disease-modifying therapeutics for the treatment of PD.

TABLE 1
Pharmacological agents inducing cellular mitophagy

| Pharmacological Class | Mitophagy inducer | Chemical structure |
|---|---|---|
| Protonophores | CCCP and FCCP | 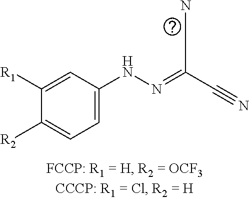 |
| | DNP | 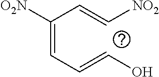 [SMILES: O=[N+]([O-])/C=C/C(=C\C=C\O)[N+](=O)[O-]] |
| K⁺ ionophores | Valinomycin | 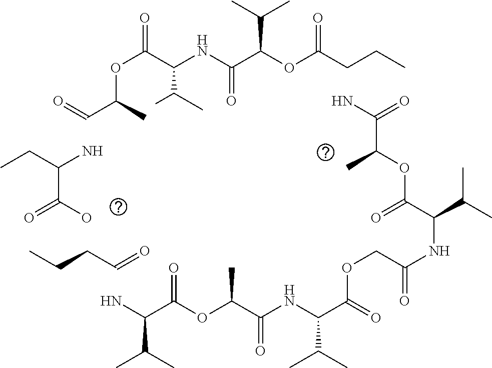 |
| | Salinomycin | 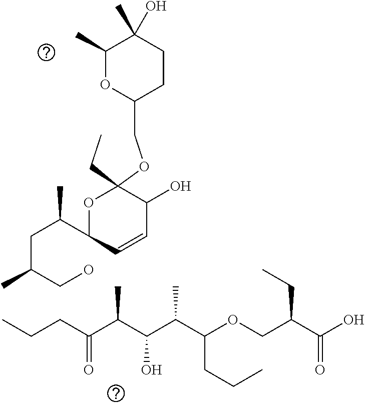 |
| Respiratory complex III and ATP synthase inhibitor | Antimycin A and oligomycin A | 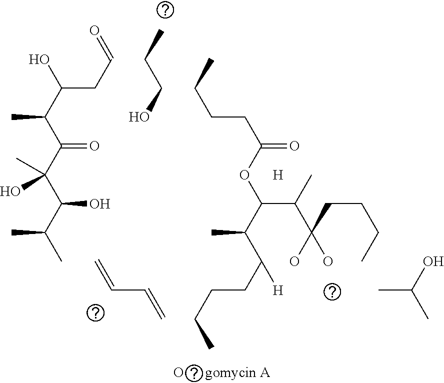 |
| | | 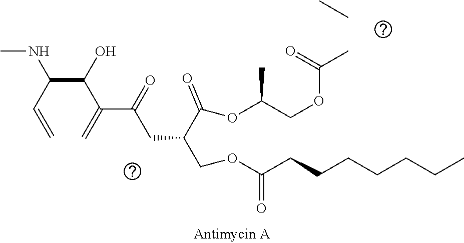 |
| Superoxide generator | Sodium selenite | 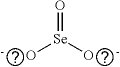 |
| Apoptotic agent | Retigeric acid B | 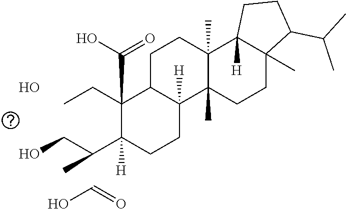 |
| Parkinsonian toxins | Diquat | 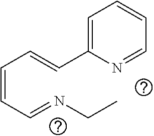 |
| | Paraquat | 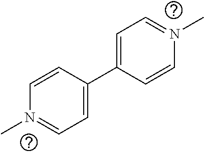 |
| | Rotenone | 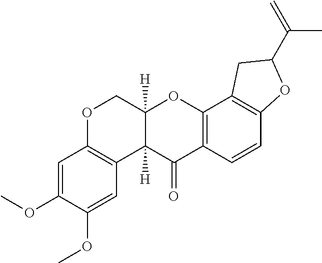 [SMILES: C=C(C)C1Cc2c(ccc3c2O[C@@H]2COc4cc(OC)c(OC)cc4[C@@H]2C3=O)O1] |
| | MPP⁺ | 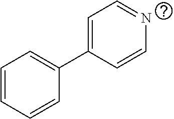 [SMILES: c1ccc(-c2ccncc2)cc1] |
| | 6-OHDA | 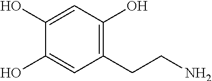 [SMILES: NCCc1cc(O)c(O)cc1O] |
| Pan-kinase inhibitor | Staurosporine | 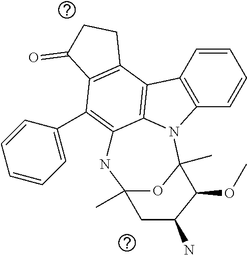 [SMILES: CO[C@H]1[C@@H](N)CC2(C)Nc3c(-c4ccccc4)c4c(c5c6ccccc6n(c35)C1(C)O2)CCC4=O] |
| Iron chelators | Deferiprone (DFP) | 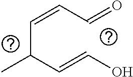 |
| | 2′,2-Bipyridyl | 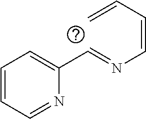 |
| | 1,10-Phenanthroline | 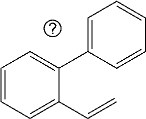 |
| | Cicloproxolamine | 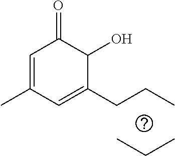 |
| PINK1 enhancer | Kinetin triphosphate, KTP | 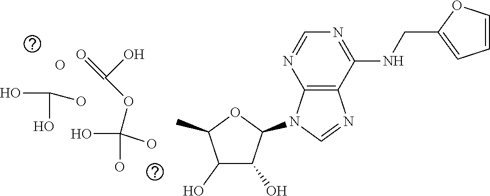 |
| p53 inhibitor | Pifithrin-α | 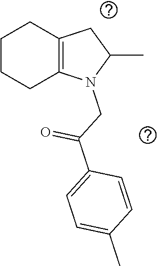 [SMILES: Cc1ccc(C(=O)CN2C3=C(CCCC3)CC2C)cc1] |
| SIRT1 agonists | Resveratrol | 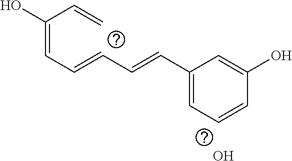 |
| | Fisetin | 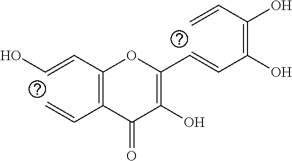 [SMILES: C=C/C(O)=C(O)\C=C\c1oc(/C=C/O)c(C=C)c(=O)c1O] |
| | SRT1720 | 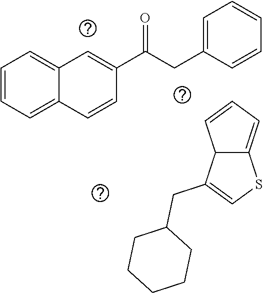 |
| NAD⁺ precursor | Nicotinamide (NAM) | 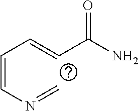 |
| PARP-1 inhibitor | AZD2281 (olaparib) | 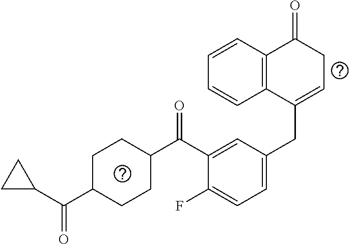 [SMILES: O=C1CC=C(Cc2ccc(F)c(C(=O)C3CCC(C(=O)C4CC4)CC3)c2)c2ccccc21] |
| Keap1 inhibitor | PMI | 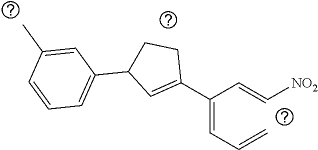 [SMILES: C=C/C=C(\C=C\[N+](=O)[O-])C1=CC(c2cccc(C)c2)CC1] |

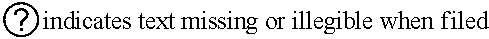

### CITATIONS FOR REFERENCES REFERRED TO IN THE SPECIFICATION

1. Kitada, T. et al. Mutations in the parkin gene cause autosomal recessive juvenile parkinsonism. Nature 392, 605-608 (1998).
2. Valente, E. M. et al. Hereditary Early-Onset Parkinson's Disease Caused by Mutations in PINK1. 304, 1158-1161 (2004).
3. Hasson, S. A. et al. High-content genome-wide RNAi screens identify regulators of parkin upstream of mitophagy. Nature 504, 291-295 (2013).
4. Ng, A. C. H., Baird, S. D. & Screaton, R. A. High-content functional genomic screening to identify novel regulators of the PINK1-parkin pathway. Methods in Enzymology 547, (Elsevier Inc., 2014).
5. Mccoy, M. K., Kaganovich, A., Rudenko, I. N., Ding, J. & Cookson, M. R. Hexokinase activity is required for recruitment of parkin to depolarized mitochondria. 23, 145-156 (2014).
6. Koch, J. C. et al. ROCK inhibition in models of neurodegeneration and its potential for clinical translation. Pharmacol. Ther. (2018). doi:10.1016/j.pharmthera.2018.03.008.
7. Wang, Y. et al. degradation by autophagy ROS-induced mitochondrial depolarization initiates PARK2/PARKIN-dependent mitochondrial degradation by autophagy. 8627, (2012).
8. Wang, Y. et al. Deubiquitinating enzymes regulate PARK2-mediated mitophagy Deubiquitinating enzymes regulate PARK2-mediated mitophagy. 8627, (2015).
9. Couzens, A. L. et al. Protein Interaction Network of the Mammalian Hippo Pathway Reveals Mechanisms of Kinase-Phosphatase Interactions. 6, (2013).
10. Cells, H. N. et al. Sequential Treatment of SH-SY5Y Cells with Retinoic Acid and Brain-Derived Neurotrophic Factor Gives Rise. (2000). doi:10.1046/j.1471-4159.2000.0750991.x
11. Sriram, S. R. et al. Familial-associated mutations differentially disrupt the solubility, localization, binding and ubiquitination properties of parkin. Hum. Mol. Genet. 14, 2571-2586 (2005).
12. Geisler, S. et al. PINK1/Parkin-mediated mitophagy is dependent on VDAC1 and p62/SQSTM1. Nat Cell Biol 12, 119-131 (2010).
13. Zhu, J. H. et al. Impaired mitochondrial biogenesis contributes to depletion of functional mitochondria in chronic MPP ρ toxicity: dual roles for ERK1/2. Cell Death Dis. 3, e312-10 (2012).
14. Georgakopoulos, N. D., Wells, G. & Campanella, M. The pharmacological regulation of cellular mitophagy. Nat. Publ. Gr. 13, 136-146 (2017).
15. Feng, Y., Lograsso, P. V., Defert, 0. & Li, R. Rho Kinase (ROCK) Inhibitors and Their Therapeutic Potential. J. Med. Chem. 59, 2269-2300 (2016).
16. Bauer, P. O., Nukina, N., Bauer, P. O. & Nukina, N. Enhanced degradation of mutant huntingtin by rho kinase inhibition is mediated through activation of proteasome and macroautophagy Enhanced degradation of mutant huntingtin by rho kinase inhibition is mediated through activation of proteasome and macroaut. 8627, (2016).
17. Gentry, E. G. et al. Rho Kinase Inhibition as a Therapeutic for Progressive Supranuclear Palsy and Corticobasal Degeneration. J. Neurosci. 36, 1316-1323 (2016).
18. Manuscript, A., Selective, H. & Kinase, R. Discovery of Substituted 4-(Pyrazol-4-yl)-phenylbenzodioxane-2-carboxamides as Potent and Highly Selective Rho Kinase (ROCK-II) Inhibitors. 51, 6642-6645 (2013).
19. Tanaka, A. et al. Proteasome and p97 mediate mitophagy and degradation of mitofusins induced by Parkin. J. Cell Biol. 191, 1367-1380 (2010).
20. Chan, N. C. et al. Broad activation of the ubiquitin-proteasome system by Parkin is critical for mitophagy. Hum. Mol. Genet. 20, 1726-1737 (2011).
21. Ding, W. et al. Parkin and Mitofusins Reciprocally Regulate Mitophagy and Mitochondrial Spheroid Formation*□. 287, 42379-42388 (2012).
22. Mclelland, G. et al. Mfn2 ubiquitination by PINK1/parkin gates the p97-dependent release of ER from mitochondria to drive mitophagy. Elife 1-35 (2018).
23. Boettcher, M. & McManus, M. T. Choosing the Right Tool for the Job: RNAi, TALEN, or CRISPR. Mol. Cell 58, 575-585 (2015).
24. Clark, I. E. et al. Drosophila pink1 is required for mitochondrial function and interacts genetically with parkin. Nature 441, 1162-1166 (2006).
25. Park, J. et al. Mitochondrial dysfunction in Drosophila PINK1 mutants is complemented by parkin. Nature 441, 1157-1161 (2006).
26. Phillips, J. P., Campbell, S. D., Michaud, D., Charbonneau, M. & Hilliker, a J. Null mutation of copper/zinc superoxide dismutase in Drosophila confers hypersensitivity to paraquat and reduced longevity. Proc. Natl. Acad. Sci. U.S.A. 86, 2761-2765 (1989).
27. Bingol, B. et al. The mitochondrial deubiquitinase USP30 opposes parkin-mediated mitophagy. Nature 509, 370-5 (2014).
28. Filograna, R. et al. Superoxide Dismutase (SOD)-mimetic M40403 Is Protective in Cell and Fly Models of Paraquat Toxicity. 291, 9257-9267 (2016).
29. Rodriguez-Perez, A. I., Dominguez-Meijide, A., Lanciego, J. L., Guerra, M. J. & Labandeira-Garcia, J. L. Inhibition of Rho kinase mediates the neuroprotective effects of estrogen in the MPTP model of Parkinson's disease. Neurobiol. Dis. 58, 209-219 (2013).
30. Tatenhorst, L. et al. Fasudil attenuates aggregation of α-synuclein in models of Parkinson's disease. Acta Neuropathol. Commun. 4, 39 (2016).
31. Frank, T. et al. Inhibition of rho kinase enhances survival of dopaminergic neurons and attenuates axonal loss in a mouse model of Parkinson's disease. (2012). doi:10.1093/brain/aws254.
32. Villar-Cheda, B. et al. Involvement of microglial RhoA/Rho-Kinase pathway activation in the dopaminergic neuron death. Role of angiotensin via angiotensin type 1 receptors. Neurobiol. Dis. 47, 268-279 (2012).
33. Chen, Y. T. et al. Bioorganic & Medicinal Chemistry Letters Chroman-3-amides as potent Rho kinase inhibitors. Bioorg. Med. Chem. Lett. 18, 6406-6409 (2008).
34. Posner, B. A., Xi, H. & Mills, J. E. J. Enhanced HTS hit selection via a local hit rate analysis. J. Chem. Inf. Model. 49, 2202-2210 (2009).
35. Yip, K. W. et al. Potential use of alexidine dihydrochloride as an apoptosis-promoting anticancer agent. Mol. Cancer Ther. 2234-2241 (2006). doi:10.1158/1535-7163.MCT-06-0134.
36. Tomitsuka, E. & Kita, K. An anticancer agent, pyrvinium pamoate inhibits the NADH fumarate reductase system—a unique mitochondrial energy metabolism in tumour microenvironments. J. Biochem. 152, 171-183 (2012).
37. Choubey, V. et al. Mutant A53T α-Synuclein induces neuronal death by increasing mitochondrial autophagy. J. Biol. Chem. 286, 10814-10824 (2011).
38. Park, Y. S., Choi, S. E. & Koh, H. C. PGAM5 regulates PINK1/Parkin-mediated mitophagy via DRP1 in CCCP-induced mitochondrial dysfunction. Toxicol. Lett. 284, 120-128 (2018).
39. Sarraf, S. A. et al. HHS Public Access. 496, 372-376 (2013).
40. Mauthe, M. et al. Chloroquine inhibits autophagic flux by decreasing autophagosome-lysosome fusion. Autophagy 14, 1435-1455 (2018).
41. Lee, J. J. et al. Basal mitophagy is widespread in Drosophila but minimally affected by loss of Pink1 or parkin. J. Cell Biol. 217, 1613-1622 (2018).
42. Narendra, D., Tanaka, A., Suen, D. F. & Youle, R. J. Parkin is recruited selectively to impaired mitochondria and promotes their autophagy. J. Cell Biol. 183, 795-803 (2008).
43. Okatsu, K. et al. Mitochondrial hexokinase HKI is a novel substrate of the Parkin ubiquitin ligase. Biochem. Biophys. Res. Commun. 428, 197-202 (2012).
44. Hosamani, R. & Muralidhara. Acute exposure of Drosophila melanogaster to paraquat causes oxidative stress and mitochondrial dysfunction. Arch. Insect Biochem. Physiol. 83, 25-40 (2013).
45. Chen, Z. et al. Genetic mosaic analysis of a deleterious mitochondrial DNA mutation in Drosophila reveals novel aspects of mitochondrial regulation and function. Mol. Biol. Cell 26, 674-684 (2015).
46. Counter, C. M. RaIA and RAIBP1 regulate mitochondrial fission at mitosis. Nat. Cell Biol. 13, 1108-1115 (2012).
47. Lieber, T., Jeedigunta, S. P., Palozzi, J. M., Lehmann, R. & Hurd, T. R. Mitochondrial fragmentation drives selective removal of deleterious mtDNA in the germline. Nature (2019). doi:10.1038/s41586-019-1213-4.

1. A screening method comprising
contacting cells expressing Parkin polypeptide comprising a wildtype or mutant Parkin polypeptide sequence, optionally labelled Parkin polypeptide comprising a wildtype or mutant Parkin polypeptide sequence and a label, with a test agent (TA);
contacting the cells with a mitophagy inducing agent (MIA) or mitophagy vehicle control; and
measuring an amount of Parkin polypeptide recruited to mitochondria in the cells contacted with the test agent compared to control cells treated with a control agent and MIA,
wherein the amount of Parkin polypeptide recruited to mitochondria in the cells contacted with the test agent compared to control cells treated with a control agent and MIA indicates whether the test agent is a putative Parkin mediated mitophagy activating or inhibiting agent.

2. The method of claim 1, wherein the cells express a suitable level of endogenous Parkin and/or wherein the cells recombinantly express labelled Parkin polypeptide comprising a wildtype or mutant Parkin polypeptide sequence and a label.

3. (canceled)

4. The method of claim 1, wherein the Parkin polypeptide is endogenous Parkin, optionally having a wildtype or mutant Parkin polypeptide sequence, and the amount of Parkin polypeptide is measured using an immunological assay, optionally with a labelled anti-Parkin antibody, optionally wherein the antibody conjugated to a fluorescent tag.

5. The method of claim 1, wherein the amount comprises determining the percentage of cells with Parkin recruited to mitochondria; and/or
wherein the measuring further comprises detecting any Parkin polypeptide in cytosol in the cells.

6. (canceled)

7. The method of claim 1, wherein the mitophagy inducing agent is selected from a proton ionophore, an iron chelator and a mitochondrial toxin.

8. The method of claim 7, wherein the proton ionophore is carbonyl cyanide m-chlorophenylhydrazone (CCCP).

9. The method of claim 1, wherein the label of labelled Parkin is selected from green fluorescent protein (GFP) including enhanced GFP and other variants, blue fluorescent protein (BFP) such as EBFP, EBFP2, Azurite, mKalama1, cyan fluorescent protein (CFP) such as ECFP, Cerulean, CyPet, mTurquoise2, and yellow fluorescent protein (YFP), such as YFP, Citrine, Venus, YPet.

10. The method of claim 1, wherein when the Parkin polypeptide comprises a mutant Parkin polypeptide sequence, the method further comprises contacting wildtype control cells expressing labelled wildtype Parkin polypeptide comprising a wildtype Parkin polypeptide and a label in one or more wells with the test agent.

11. The method of claim 1, wherein the MIA is added to cells in an amount to cause endogenous and/or Parkin polypeptide, optionally labelled Parkin polypeptide, in control cells to experience greater than or about 60% and 80%.

12. The method of claim 1, wherein the test agent is assessed as a mitophagy inhibiting agent if the Parkin polypeptide experiences at least 10% less than a control treated with control agent and/or MIA; or
wherein the test agent is assessed as a mitophagy activating agent if the Parkin polypeptide experiences at least 10% greater than a control treated with control agent and/or MIA.

13. (canceled)

14. The method of claim 1, wherein the Parkin polypeptide, optionally the labelled Parkin polypeptide, comprises a mutant Parkin polypeptide sequence and comprises one or more mutations selected from K161N, T240R, W403A and G430D, or wherein the Parkin polypeptide is endogenous Parkin polypeptide and the cells exhibit reduced mitophagy.

15. (canceled)

16. The method of claim 1, wherein the amount of the Parkin polypeptide, optionally labelled Parkin polypeptide, recruitment to mitochondria in the cells contacted with the test agent and the control cells is measured by or using immunofluorescence; and/or
wherein the method is performed using control cells expressing labelled wildtype Parkin polypeptide
wherein the amount of labelled wildtype Parkin polypeptide recruited to mitochondria in cells treated with the control agent is compared to the amount of mutant Parkin polypeptide recruited to mitochondria in cells treated with the control agent.

17. (canceled)

18. The method of claim 1, wherein the cells are contacted with the test agent for at least 30 min, 45 min, 60 min, 75 min, 90 min, 105 min or 120 min before contacting with the mitophagy inducing agent; and/or
wherein the test agent is at a concentration of 0.4 μM to 40 μM, preferably 1 μM to 10 μM, more preferably 2 μM to 4 μM, most preferably 4 μM.

19. The method of claim 1, wherein the cells are HEK293 cells.

20.-22. (canceled)

23. The method of claim 1, wherein viability of the cells is assessed after the cells are contacted with the test agent.

24. The method of claim 1, wherein the amount of Parkin polypeptide, optionally labelled Parkin polypeptide, recruited to mitochondria in cells contacted with the test agent and the control cells is determined by:
i) segmenting the cell based on Parkin polypeptide, optionally labelled Parkin polypeptide, and a cell/nucleus defining stain such as DAPI stain to identify individual cells, and
ii) classifying each cell of the cells according to whether the cell contains mitochondrial Parkin, which is assessed by determining if the cell comprises fluorescent Parkin signal abutting a signal of the mitochondrial detection agent, or lacks fluorescent Parkin signal abutting a signal of the mitochondrial detection agent.

25. The method of claim 1, wherein the amount of Parkin polypeptide, optionally labelled Parkin polypeptide, recruited to mitochondria in the cells contacted with the test agent and the control cells is determined by high content imaging image acquisition software.

26. (canceled)

27. The method of claim 1, wherein the cells are grown on a cell-adherence agent coated surface comprising poly-lysine prior to contacting the cells with the test agent and the cells are allowed to adhere to the cell-adherence agent coated surface for 24 hours prior to contacting with the test agent.

28.-29. (canceled)

30. The method of claim 1, wherein the test agent is a molecule from a small molecule library.

31.-32. (canceled)

33. A high-throughput screening assay comprising
preparing a plurality of wells comprising test wells and control wells each comprising cells expressing Parkin polypeptide comprising a wildtype or mutant Parkin polypeptide sequence, optionally wherein the Parkin polypeptide is labelled Parkin polypeptide comprising a wildtype or mutant Parkin polypeptide sequence and a label,
contacting the plurality of test wells with a plurality of test agents (TAs) and optionally one or more of the control wells with a control agent,
contacting a subset of the plurality of test wells and control wells with either a mitophagy inducing agent (MIA) or a mitophagy vehicle control,
measuring an amount of Parkin polypeptide recruited to mitochondria in the plurality of test wells and the control wells,
comparing the amount of Parkin polypeptide recruited to mitochondria in the plurality of test wells contacted with TA and MIA to the amount of Parkin polypeptide recruited to mitochondria in the control wells treated with MIA and optionally a control agent, wherein
a TA that increases Parkin recruitment to the mitochondria compared to control wells is a putative Parkin mediated mitophagy activating agent and a TA that decreases Parkin recruitment to the mitochondria compared to control wells is a putative Parkin mediated mitophagy inhibiting agent.

34. A high-throughput screening assay comprising:
preparing a plurality of test wells and control wells each comprising cells recombinantly expressing labelled Parkin polypeptide comprising a wildtype or a mutant Parkin polypeptide sequence and a label,
contacting the plurality of test wells with a plurality of test agents
contacting the plurality of test wells with either a mitophagy inducing agent (MIA) or a mitophagy vehicle control, wherein one or more of the plurality of test wells are contacted with the MIA or the mitophagy vehicle control,
measuring an amount of labelled Parkin polypeptide recruited to mitochondria in the plurality of test wells,
comparing the amount of labelled Parkin polypeptide recruited to mitochondria in the plurality of test wells contacted with TA to the amount of labelled Parkin polypeptide recruited to mitochondria in the plurality of test wells contacted with the control agent, and
comparing the amount of labelled Parkin polypeptide recruited to mitochondria in the plurality of test wells contacted with MIA to the amount of labelled Parkin polypeptide recruited to mitochondria in the plurality of test wells contacted with the mitophagy vehicle control.

35. The assay of claim 33, wherein the method further comprises comparing the amount of Parkin polypeptide recruited to mitochondria in the plurality of test wells contacted with MIA to the amount of labelled Parkin polypeptide recruited to mitochondria in the plurality of test wells contacted with the mitophagy vehicle control.

36. The assay of claim 33, wherein the high-throughput screening assay comprises using cells recombinantly expressing labelled Parkin or comprises using cells expressing endogenous Parkin, optionally wherein the labelled Parkin comprises a mutant Parkin polypeptide sequence and a label.

37.-38. (canceled)

39. The method of claim 1 further comprising calculating a Z score and/or staining the cells with a nuclear dye optionally DAPI.

40.-41. (canceled)
